(19) United States
(12) Patent Application Publication
(10) Pub. No.: US 2014/0024540 A1
(43) Pub. Date: Jan. 23, 2014

(54) LIGAND IDENTIFICATION

(71) Applicants: Renata Pasqualini, Houston, TX (US); Wadih Arap, Houston, TX (US); Ricardo J. Giordano, Sao Paulo (BR); Emmanuel Dias-Neto, Sao Paulo (BR); Diana N. Nunes, Sao Paulo (BR)

(72) Inventors: Renata Pasqualini, Houston, TX (US); Wadih Arap, Houston, TX (US); Ricardo J. Giordano, Sao Paulo (BR); Emmanuel Dias-Neto, Sao Paulo (BR); Diana N. Nunes, Sao Paulo (BR)

(73) Assignee: BOARD OF REGENTS, THE UNIVERSITY OF TEXAS SYSTEM, Austin, TX (US)

(21) Appl. No.: 13/994,165

(22) PCT Filed: Dec. 17, 2010
(86) PCT No.: PCT/US10/61129

§ 371 Date: Oct. 10, 2013

(51) Int. Cl. C12N 15/10

(52) U.S. Cl. CPC C12N 15/1037
USPC 506/2; 506/9

(57) ABSTRACT

The present disclosure provides new and improved phage display methodologies. Among other things, the present invention provides methodologies that do not utilize bacterial amplification of phage. Alternatively or additionally, the present invention provides phage display systems that rapidly identify ligands. Alternatively or additionally, the present invention provides phage display methodologies for use in human subjects. Alternatively or additionally, the present invention provides phage display systems that allow detection and/or characterization of low abundance ligands.

### GOVERNMENT SUPPORT

The United States Government has provided grant support utilized in the development of the present invention. In particular, grants from the National Institutes of Health and the U.S. Department of Defense have supported development of this invention. The United States Government may have certain rights in the invention.

### BACKGROUND OF THE INVENTION

Combinatorial phage display has been used in the last 20 years in the identification of protein-ligands and protein-protein interactions, uncovering relevant molecular recognition events. Because of the strong predictive value of functional relationships revealed by specific protein interactions, peptide-protein or antibody-antigen pairs selected from phage display libraries serve as potential reagents in a vast range of biomedical and translational applications.

Conventional phage display selection typically starts with exposure of a library to targets of interest, followed by recovery by infection and amplification in host bacteria, which allow viral multiplication and generate thousands of newly-formed phage particles. Upon plating of the host bacteria, transducing-units (TU) are quantified. Amplified phage populations serve for additional selection round(s) and allow enrichment of selective clones, which may be determined by comparing unselected to selected libraries through DNA sequencing [1]-[4]. Two of the most rate-limiting steps of combinatorial phage display library selection are (i) the counting of transducing units and (ii) the sequencing of the encoded displayed ligands.

Conventional phage display approaches have long provided biomedical findings of value. The present invention, however, recognizes that this conventional methodology includes many practical limitations and in particular includes limitations that restrict its relevance in large scale environments.

### SUMMARY OF THE INVENTION

The present disclosure provides new and improved phage display methodologies. Among other things, the present invention provides methodologies that do not utilize bacterial amplification of phage. Alternatively or additionally, the present invention provides phage display systems that rapidly identify ligands. Alternatively or additionally, the present invention provides phage display methodologies for use in human subjects. Alternatively or additionally, the present invention provides phage display systems that allow detection and/or characterization of low abundance ligands.

### BRIEF DESCRIPTION OF THE DRAWING

FIG. 1. Exemplary data demonstrating a comparison of TU-counting and qPhage. (A) Schematic representation of phage genome and relative location of the cloning site and two sets of primers used. Primer set #1 targets the Tet^{R} gene and was used for quantification with real-time PCR; primer set #2 flanks the insert coding for the peptide displayed in pill, and served for large-scale sequencing (B and C, respectively). TU-counting and qPhage titration output [determined by cycle-thresholds (Ct)]; note the limited quantification range for TU-counting relative to qPhage. Comparative results of TU-counting and qPhage titration of Fd-tet (D) and RGD-4C phage (E) are shown. Asterisk indicates that the high bacterial density prevented accurate TU determination.

FIG. 2. Exemplary data demonstrating phage binding and internalization assays. Binding of αv integrin-binding ligand phage (displaying RGD-4C) or insertless phage to endothelial cells. Quantification by conventional TU-counting (A) and qPhage (B) are shown. For internalization, endothelial cells were incubated with RGD-4C phage or insertless phage for short (5 minutes) or long (ON) incubation. Internalized phage particles were detected by immunostaining (C) or qPhage (D).

FIG. 3. Depiction of overlap of sequences revealed by Sanger sequencing and next-generation pyrosequencing. Venn diagrams represent the peptides revealed by Sanger sequencing (purple) and large-scale next-generation pyrosequencing (salmon) in tissues such as bone marrow, fat, muscle, skin (A) or the non-selected CX7C library (B). The dark-pink area represents sequences found by both approaches. Numbers indicate the total peptides in each group and their percentage relative to TU-counting (purple and overlap areas) or relative to next-generation sequencing (salmon-colored area). No overlap was seen for the non-administered phage library sequences, produced by both methods. Circle sizes are proportional to the number of sequences revealed by each strategy.

FIG. 4. Exemplary data depicting saturation plots of peptide diversity coverage after next-generation sequencing. The plot shows the number of distinct peptides observed in bone marrow (A), fat (B), muscle (C), skin (D) or the non-selected library (E), as a function of the total number of sequences evaluated for each tissue after filtering. All tissues investigated attained or nearly attained saturation (as determined by the predicted number of distinct peptides in each tissue), whereas nothing approaching saturation was observed for the unselected library (straight line).

FIG. 5. Exemplary depiction of analysis of cost and time required to generate phage sequences using Sanger- or 454-pyrosequencing methods. Cost (A) and time (B) to generate sequences with Sanger-sequencing of individual TU (red) versus DNA amplification (qPhage) followed by next-generation sequencing (blue). Data and estimates used for this analysis are presented in Table 4.

FIG. 6. Exemplary data depicting the amino acid frequency association between Sanger and next-generation pyrosequencing phage. Data was evaluated by applying a chi-square test, which indicated a significant association between both methods (p<0.001). Pearson correlation analysis indicated a strong positive correlation between sequences obtained by both approaches (r=0.996, p<0.001).

FIG. 7. Exemplary data depicting enriched motifs revealed by Sanger-sequencing and next-generation sequencing. The graph shows the number of distinct, statistically significant (Fisher's exact test, one-sided, P<0.05) tri, tetra, and penta amino acid motifs enriched in all tissues, after their frequencies were compared between every target tissue and the non-selected phage display library. Motifs derived from Sanger-sequencing are shown in the white bars.

### DEFINITIONS

Antibody: As used herein, the term “antibody” is intended to include immunoglobulins and fragments thereof which are specifically reactive to the designated protein or peptide, or fragments thereof. Suitable antibodies include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, antibody-like molecules, and antibody fragments. As used herein, the term “antibodies” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. Antibody polypeptides for use herein may be of any type (e.g., IgA, IgD, IgE, IgG, IgM).

Antibody fragment: As used herein, an “antibody fragment” includes a portion of an intact antibody, such as, for example, the antigen-binding or variable region of an antibody. Examples of antibody fragments include Fab, Fab′, F(ab′)2, Fc and Fv fragments; triabodies; tetrabodies; linear antibodies; single-chain antibody molecules; and multi specific antibodies formed from antibody fragments. The term “antibody fragment” also includes any synthetic or genetically engineered protein that acts like an antibody by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region.

Determine: Many methodologies described herein include a step of “determining”. Those of ordinary skill in the art, reading the present specification, will appreciate that such “determining” can utilize any of a variety of techniques available to those skilled in the art, including for example specific techniques explicitly referred to herein. In some embodiments, a determination involves manipulation of a physical sample. In some embodiments, a determination involves consideration and/or manipulation of data or information, for example utilizing a computer or other processing unit adapted to perform a relevant analysis. In some embodiments, a determination involves receiving relevant information and/or materials from a source.

Host: The term “host” is used herein to refer to a system (e.g., a cell, organism, etc.) in which a nucleic acid or polypeptide of interest is present. In some embodiments, a host is a system that expresses a particular polypeptide of interest.

Isolated: The term “isolated”, as used herein, refers to an agent or entity that has either (i) been separated from at least some of the components with which it was associated when initially produced (whether in nature or in an experimental setting); or (ii) produced by the hand of man. Isolated agents or entities may be separated from at least about 10%, at least about 20%, at least about 30%, at least about 40%, at least about 50%, at least about 60%, at least about 70%, at least about 80%, at least about 90%, or more of the other components with which they were initially associated. In some embodiments, isolated agents are more than 90%, 91%, 92%, 93%, 94%, 95%, 96%, 97%, 98%, 99% pure.

Library: As used herein, the term “library” refers to a collection of molecules. A library can contain a few or a large number of different molecules, varying from about two to about 10¹⁵ molecules or more. The chemical structure of the molecules of a library can be related to each other or be diverse. If desired, the molecules constituting the library can be linked to a common or unique tag, which can facilitate recovery and/or identification of the molecule. In some embodiments, a library contains a plurality of ligand-encoding phage.

Low abundance: As used herein, the term “low abundance” when used in reference to a ligand refers to a ligand that i) is present in low concentration of putative ligands and/or ii) is specifically localized to and present at low prevalence in one or more organs or tissues in a primate subject to which a library of ligand-encoding phage has been administered.

Nucleic acid molecule: The term “nucleic acid molecule” is used broadly to mean any polymer of two or more nucleotides, which are linked by a covalent bond such as a phosphodiester bond, a thioester bond, or any of various other bonds known in the art as useful and effective for linking nucleotides. Such nucleic acid molecules can be linear, circular or supercoiled, and can be single stranded or double stranded, e.g. single stranded or double stranded DNA, RNA or DNA/RNA hybrid. In some embodiments, nucleic acid molecules are or include nucleic acid analogs that are less susceptible to degradation by nucleases than are DNA and/or RNA. For example, RNA molecules containing 2′-O-methylpurine substitutions on the ribose residues and short phosphorothioate caps at the 3′- and 5′-ends exhibit enhanced resistance to nucleases (Green et al., Chem. Biol., 2:683-695 (1995), which is incorporated herein by reference). Similarly, RNA containing 2′-amino-2′-deoxypyrimidines or 2′-fluoro-2′-deoxypyrimidines is less susceptible to nuclease activity (Pagratis et al., Nature Biotechnol., 15:68-73 (1997), which is incorporated herein by reference). Furthermore, L-RNA, which is a stereoisomer of naturally occurring D-RNA, is resistant to nuclease activity (Nolte et al., Nature Biotechnol., 14:1116-1119 (1996); Klobmann et al., Nature Biotechnol., 14:1112-1115 (1996); each of which is incorporated herein by reference). Such RNA molecules and methods of producing them are well known in the art and can be considered to be routine (see Eaton and Piekern, Ann. Rev. Biochem., 64:837-863 (1995), which is incorporated herein by reference). DNA molecules containing phosphorothioate linked oligodeoxynucleotides are nuclease resistant (Reed et al., Cancer Res. 50:6565-6570 (1990), which is incorporated herein by reference). Phosphorothioate-3′hydroxypropylamine modification of the phosphodiester bond also reduces the susceptibility of a DNA molecule to nuclease degradation (see Tam et al., Nucl. Acids Res., 22:977-986 (1994), which is incorporated herein by reference).

Organ or Tissue: As used herein, the terms “organ or tissue” and “selected organ or tissue” are used in the broadest sense to mean an organ or tissue in or from a body. In some embodiments, an organ or tissue has a pathology, for example, lung containing lung tumors, whether primary or metastatic lesions. In some embodiments, an organ or tissue is normal. The term “control organ or tissue” is used to mean an organ or tissue other than a selected organ or tissue of interest. In some embodiments, a control organ or tissue is characterized by the inability of a ligand-encoding phage to home to the control organ or tissue and, therefore, is useful for identifying selective binding of a molecule to a selected organ or tissue.

Polypeptide: A “polypeptide”, generally speaking, is a string of at least two amino acids attached to one another by a peptide bond. In some embodiments, a polypeptide includes at least 3-5 amino acids, each of which is attached to others by way of at least one peptide bond. Those of ordinary skill in the art will appreciate that, in some embodiments, polypeptides include one or more “non-natural” amino acids or other entities that nonetheless are capable of integrating into a polypeptide chain. In some embodiments, a polypeptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues.

Predominantly present: The term “predominantly present”, as used herein to refer to amino acid residues in a polypeptide, refers to the presence of the residue at a particular location across a population. In some embodiments, an amino acid is considered to be predominantly present if, across a population of polypeptides, the particular amino acid is statistically present in at least about 50%, about 55%, about 60%, about 65%, about 70%, about 75%, about 80%, about 85%, about 90%, about 95%, about 96%, about 97%, about 98%, about 99% or more of the polypeptides.

Sample: As used herein, the term “sample” refers to a cell, tissue, organ or portion thereof that is isolated from a body. It will be appreciated that a sample may be or comprise a single cell or a plurality of cells. In some embodiments, a sample is or comprises a histologic section or a specimen obtained by biopsy (e.g., surgical biopsy); in some embodiments, a sample is or comprises cells that are or have been placed in or adapted to tissue culture. In some embodiments, a sample is a specimen obtained from a dead body (e.g., by autopsy). In some embodiments, the sample is or comprises an intact organ or tissue.

Sample processing: As used herein, the term “sample processing” generally refers to various steps that may be accomplished to prepare a sample for quantification. In some embodiments, crude sample (e.g., whole tissue, homogenized tissue, etc.) is prepared. In some embodiments, purified or highly purified sample is prepared.

Specificity: As is known in the art, “specificity” is a measure of the ability of a particular ligand (e.g., a ligand encoded by a phage) to distinguish its binding partner (e.g., a target tissue, or organ of interest) from other potential binding partners (e.g., a control tissue or organ).

Subject: As used herein, the terms “subject,” “individual” or “patient” refer to a human or a non-human mammalian subject. In some embodiments, a subject is a non-human primate. In some embodiments, a subject is a human. In some embodiments, a human subject is a patient having a surgical tumor resection or a surgical biopsy. In some embodiments, a human subject is a patient suffering from brain death or trauma. In some embodiments, a human subject is an end-of-life patient.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues that share one or more structural and/or functional characteristics. For example, as is well known by those of ordinary skill in the art, certain amino acids are typically classified as “hydrophobic” or “hydrophilic” amino acids, and/or as having “polar” or “non-polar” side chains In some embodiments, substitution of one amino acid for another of the same type is considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic | Asp | D | polar | negative | −3.5 |
| acid | | | | | |
| Cysteine | Cys | C | nonpolar | neutral | 2.5 |
| Glutamic | Glu | E | polar | negative | −3.5 |
| acid | | | | | |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying identical sequences, the programs mentioned above typically provide an indication of the degree of identity. In some embodiments, two sequences are considered to be substantially identical if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are identical over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Therapeutic agent: As used herein, the phrase “therapeutic agent” refers to any agent that elicits a desired biological or pharmacological effect.

Treatment: As used herein, the term “treatment” refers to any method used to alleviate, delay onset, reduce severity or incidence, or yield prophylaxis of one or more symptoms or aspects of a disease, disorder, or condition. For the purposes of the present invention, treatment can be administered before, during, and/or after the onset of symptoms.

As used in this application, the terms “about” and “approximately” are used as equivalents. Any numerals used in this application with or without about/approximately are meant to cover any normal fluctuations appreciated by one of ordinary skill in the relevant art.

### DETAILED DESCRIPTION OF CERTAIN EMBODIMENTS OF THE INVENTION

The present disclosure provides new and improved phage display methodologies. Among other things, the present invention provides methodologies that do not utilize bacterial amplification of phage. Alternatively or additionally, the present invention provides phage display systems that rapidly identify ligands. Alternatively or additionally, the present invention provides phage display methodologies for use in human subjects. Alternatively or additionally, the present invention provides phage display systems that allow detection and/or characterization of low abundance ligands.

### Phage Display

For over two decades, phage display has been used to identify relevant protein interaction and recognition sites in receptor-ligand and antigen-antibody binding systems. Because of the strong predictive value of functional relationships revealed by specific protein interactions, peptide-protein or antibody-antigen pairs selected from phage display libraries serve as potential reagents in a vast range of biomedical and translational applications [1]-[4] U.S. Pat. No. 6,933,281 and US Publication No. 20080124277 describe exemplary phage display methodologies, the entire contents of each of which is incorporated herein by reference.

A conventional phage display selection typically starts with exposure of a library to targets of interest in vitro or in vivo. After the unbound and non-specific binding populations are removed, the remaining phage particles are recovered by infection and amplification in host bacteria growing in medium under either a selective genetic pressure (such as antibiotic resistance) or a differential identifying color scheme. Host bacteria allow viral multiplication and generate thousands of newly-formed phage particles. Upon plating of the host bacteria, lysogenic phage (i.e., non-lytic M13-derived) yields bacterial colonies whereas lytic phage (i.e., Lambda-derived) generates plaques in a bacterial lawn; each resulting bacterial colony or phage plaque is considered a transducing-unit (TU). Amplified phage populations serve for additional selection round(s) and allow enrichment of selective clones, which may be determined by comparing unselected to selected libraries through DNA sequencing [1]-[4]. Two of the most labor- and cost-intensive steps in conventional phage display selection are (i) the counting of transducing units and (ii) the phage DNA sequencing (from each individual colony or plaque) to determine the corresponding peptide sequence of the encoded ligand(s).

Conventional phage display approaches have long provided biomedical findings of value. The present invention, however, recognizes that this conventional methodology includes many practical limitations and in particular includes limitations that restrict its relevance in large scale environments.

The present invention provides new and improved phage display methodologies. Among other things, the present invention provides methodologies that do not utilize bacterial amplification of phage. Among other things, avoidance of a bacterial amplification steps allows identification of ligands which are missed in conventional phage display systems, for example because of non-infective phage or low abundance of phage binding to a target organ or tissue. In some embodiments, phage display library screening methodology in accordance with the present invention quantifies non-infective phage particles present in the sample.

In some embodiments, the present invention provides improvements to certain phage display methodologies, which improvement comprises, for example, obtaining an organ or tissue sample from an autopsy, quantifying ligands encoded by phage in the absence of bacteria, quantifying ligands encoded by phage by quantitative real time PCR, identifying ligands present at low abundance in the one or more target organs or tissues, and/or obtaining sequence information by next generation pyrosequencing.

Alternatively or additionally, the present invention provides phage display systems that rapidly identify ligands. In some embodiments, samples are obtained within a time period between 1 h and 72 h (e.g., 1, 2, 3, 4, 5, 6, 7, 8, 9, 10, 11, 12, 13, 14, 15, 16, 17, 18, 19, 20, 21, 22, 23, 24, 36, 48, 60, or 72 hours) after administration of a phage display library to a subject. In some embodiments, steps of obtaining a sample and quantifying phage encoded ligands are completed within a time period not longer than 48 hours (e.g., not longer than 1, 2, 3, 4, 5, 6, 7, 8, 9, 10, 11, 12, 24, 36, or 48 hours).

Alternatively or additionally, the present invention provides phage display systems that allow detection and/or characterization of low abundance ligands.

In general, provided methods often comprise steps of 1) obtaining a sample of a target organ or tissue of interest from a mammalian subject (e.g., primate subject such as monkey or human) to whom a library of ligand-encoding phage has been administered, and 2) quantifying by quantitative PCR phage content from the sample. In some embodiments, methods provided by the present disclosure include a step of determining nucleotide sequence information for at least one ligand encoded by the phage from the sample. In some embodiments, provided methods do not require bacterial amplification of phage.

### Phage Display Libraries

A phage display library in accordance with the present disclosure contains a collection of phage, each of which displays on its surface at least one ligand (e.g., a putative targeting ligand). In some embodiments, a phage display library contains phage that express two or more different ligands. In some embodiments, a phage display library contains phage that express up to 10¹⁵ more different ligands.

In some embodiments, ligands expressed by phage in a phage display library are polypeptide ligands. In some such embodiments, ligands expressed by phage in a phage display library are polypeptides at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 55, 60, 65, 70, 75, 80, 85, 90, 95, 100, 110, 120, 130, 140, 150, 160, 170, 180, 190, 200, 250, 300, 350, 400, 450, 500 or more amino acids in length. In some such embodiments, ligands expressed by phage in a phage display library are antibodies or antibody fragments. In some such embodiments, ligands expressed by phage in a phage display library are peptides whose length is within a range having a lower bound of about 3, 4, 5, 6, 7, 8, 9, 10, 11, 12, 13, 14, 15, 16, 17, 18, 19, or 20 amino acids and an upper bound of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. In some embodiments, such a peptide has a length within the range of about 3 to 100 amino acids in length. For example, in some particular embodiments, peptide ligands are about 5-20, about 5-15, about 6-10, or about 7-9 amino acids in length.

In some embodiments, a phage display library includes one or more phage expressing a ligand attached to a “tag”. In general, a tag is a moiety that facilitates recovery and/or identification of the ligand to which it is attached. Useful such tags include physical, chemical or biological moieties, for example such as polypeptide moieties, plastic or metallic microbeads, oligonucleotide moieties, bacteriophage moieties, etc. A wide variety of such tags, and of methods for linking them with appropriate ligands for use in a library as described herein, are known in the art (see, for example, Hermanson, Bioconjugate Techniques, Academic Press 1996, which is incorporated herein by reference). The link between a ligand and a tag can be a covalent or a non-covalent bond and, if desired, the link can be selectively cleavable from the ligand.

A phage display library is typically a collection of phage that have been genetically engineered to express a set of ligands (e.g., putative targeting ligands) on their outer surface. In some embodiments, DNA sequences encoding ligands are inserted in frame into a gene encoding a phage capsule protein. In some embodiments, DNA sequences encoding ligands are inserted into a vector (to give but one example, a fUSE5 vector) that includes regulatory elements ensuring and/or controlling expression in phage.

In some embodiments, at least some ligands (e.g., putative targeting ligands) within a phage display library share one or more common sequence elements in that, for at least a set of ligands within the library, all ligands within the set have the same amino acid at one or more particular designated positions, and commonly at at least 2, 3, 4, 5, 6, 7, 8, 9, 10, 11, 12, 13, 14, 15, 16, 17, 18, 19, 20 or more such positions. In some embodiments, all ligands within such a set have the same amino acids at the same positions for least 40%, 45%, 50%, 55%, 60%, 65%, 70%, 75%, 80%, 85%, 90%, 95%, 96%, 97%, 98%, 99% or more of their residues. In some embodiments, different ligands within such a set have different amino acids, when compared to one another, in at least one position. In some embodiments, for at least one position, at least 2, 3, 4, 5, 6, 7, 8, 9, 10, 11, 12, 13, 14, 15, 16, 17, 18, 19, 20 or more different amino acids are represented at a particular position in the relevant set of ligands. In some embodiments, for at least one position, all 20 natural amino acids are represented at a particular position in the relevant set of ligands. In some embodiments, for at least one position at which different ligands in the set differ in sequence, fewer than all possible residues are represented across the set.

In some embodiments, a library comprises a set of ligands that share a common sequence element comprising at least two fixed residues, that are identical in all members of the set and that flank one or more resides that vary across the set. show one or more sequence differences when compared to one another. In some such embodiments, at least one of the fixed residues is or comprises a cysteine; in some such embodiments, both fixed residues are or comprise a cysteine. In some such embodiments, the fixed residues flank a sequence that is 1, 2, 3, 4, 5, 6, 7, 8, 9, 10, 11, 12, 13, 14, 15, 16, 17, 18, 19, 20 or more amino acids in length.

It will be appreciated that a phage display library can encode and/or express any variety of ligands, including, for example, polypeptides, peptoids, peptidomimetics, antibodies, antibody fragments, nucleotides, etc., and combinations thereof.

A variety of methods for preparing libraries as described herein are well known in the art and various libraries are commercially available (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994), each of which is incorporated herein by reference). In some embodiments, ligands displayed by phage within a library are produced in vitro; in some embodiments, ligands are expressed from a nucleic acid, which can be produced in vitro. Methods of synthetic peptide and nucleic acid chemistry are well known in the art.

In some embodiments, production of ligands in a library comprises constructing a cDNA expression library from mRNA from a source (e.g., a cell, tissue, organ or organism) of interest. Methods for producing a cDNA expression library are well known in the art (see, for example, Sambrook et al., Molecular Cloning: A laboratory manual (Cold Spring Harbor Laboratory Press 1989), which is incorporated herein by reference).

In some embodiments, one or more ligands in a phage display library is or comprises a nucleic acid molecule (e.g., DNA, RNA or analogs thereof). A wide variety of methods are known in the art for preparing or providing nucleic acid molecules for display in a phage display library. To give but a few examples, cDNA can be constructed from mRNA (e.g., collected from a cell, tissue, organ or organism of interest); genomic DNA can be isolated from one or more cells; RNA can be collected from cells or can be chemically synthesized. In some embodiments, nucleic acids are chemically synthesized; chemical synthesis can facilitates the production of randomized regions in the molecules. If desired, the randomization can be biased to produce a library of nucleic acid molecules containing particular percentages of one or more nucleotides at a position in the molecule (U.S. Pat. No. 5,270,163, issued Dec. 14, 1993, which is incorporated herein by reference). In some embodiments, nucleic acids can be fragmented, for example using enzymatic (e.g., with restriction enzymes) or chemical fragmentation.

### Library Administration

In general, a library can be administered to a subject by any available method. For example, in some embodiments, a library is administered by injecting the library into the circulation of the subject such that the molecules pass through the selected organ or tissue; after an appropriate period of time, circulation is terminated, for example, by perfusion through the heart or by removing a sample of the organ or tissue (U.S. Pat. No. 5,622,699, supra, 1997; see, also, Pasqualini and Ruoslahti, Nature 380:364-366 (1996), which is incorporated herein by reference). Alternatively, a cannula can be inserted into a blood vessel in the subject, such that the library is administered by perfusion for an appropriate period of time, after which the library can be removed from the circulation through the cannula or the subject can be sacrificed or anesthetized to collect an organ or tissue sample. A library also can be shunted through one or a few organs or tissues including a selected organ or tissue, by cannulation of the appropriate blood vessels in the subject. It is recognized that a library also can be administered to an isolated perfused organ or tissue.

Typically, some time after administration of a phage display library to a subject, selected organ or tissue is collected, phage content in the selected organ or tissue is quantified, and in some cases, ligands present in the selected organ or tissue are identified. If desired, one or more control organs or tissues or a part of a control organ or tissue are sampled as well. In some embodiments, after administration of a phage display library to a subject, selected organ or tissue is collected and phage content is not quantified. In some embodiments, after administration of a phage library to a subject, selected organ or tissue is collected and ligands present in the selected organ or tissue are identified (e.g., by sequencing).

It will be appreciated that a subject to whom a phage display library is administered may be a human or a non-human mammalian subject. In one aspect, the present invention provides teachings relevant to localization of ligands in primate (e.g., human) subjects. In some embodiments, a subject is a non-human primate. In some embodiments, a subject is a human. In some embodiments, a human subject is a patient having a surgical tumor resection or a surgical biopsy. In some embodiments, a human subject is a patient suffering from brain death or trauma. In some embodiments, a human subject is an end-of-life patient.

Once a library is administered to a subject, selective localization of individual library members to tissues and/or organs of interest in the subject can be assessed. Selective localization may be determined, for example, by methods disclosed below, wherein the administration to a subject of a library of such phage that have been genetically engineered to express a multitude of such targeting ligands of different amino acid sequence is followed by collection of one or more organs, tissues or cell types from the subject and identification of phage found in that organ, tissue or cell type. A phage expressing a targeting ligand sequence is considered to be selectively localized to a tissue or organ if it exhibits greater binding in that tissue or organ compared to a control tissue or organ. In some embodiments, selective localization of a targeting peptide results in a two-fold, three-fold, four-fold, five-fold, six-fold, seven-fold, eight-fold, nine-fold, ten-fold or higher enrichment in or higher enrichment of the phage in the target organ, tissue or cell type, compared to a control organ, tissue or cell type. In some embodiments, a phage expressing a targeting ligand that exhibits selective localization shows an increased enrichment in the target organ compared to a control organ when phage recovered from the target organ are reinjected into a second host for another round of screening. Further enrichment may be exhibited following a third round of screening. In some embodiments, phage expressing a putative target ligand exhibit a two-fold, a three-fold or higher enrichment in the target organ compared to control phage that express a non-specific peptide or that have not been genetically engineered to express any putative target peptides. In some embodiments, localization to a target organ of phage expressing the target ligand is at least partially blocked by co-administration of a synthetic ligand containing the target ligand sequence.

### Sample

In general, selective localization is assessed by collection of one or more samples from the subject to which the library was administered.

It will be appreciated that a sample in accordance with the present disclosure may be a cell, tissue, organ or portion thereof, or any combination thereof that is isolated from the body of the subject. It will be appreciated that a sample may be or comprise a single cell or a plurality of cells. In some embodiments, a sample is or comprises a histologic section or a specimen obtained by biopsy (e.g., surgical biopsy); in some embodiments, a sample is or comprises cells that are or have been placed in or adapted to tissue culture. In some embodiments, a sample is or comprises a specimen obtained from a dead body (e.g., by autopsy). In some embodiments, a sample is or comprises an intact organ or tissue.

In some embodiments, a sample is obtained from a subject (e.g., a human patient) having a surgical tumor resection or a surgical biopsy. In some embodiments, a sample is obtained from a subject (e.g., a human patient) suffering from brain death or trauma. In some embodiments, a sample is obtained from a subject (e.g., a human patient) that is an end-of-life patient.

A sample may be obtained from any target organ or tissue of interest. It will be appreciated that a target organ or tissue may be a normal organ or tissue or an organ or tissue having a pathology, for example, lung containing lung tumors, whether primary or metastatic lesions. In some embodiments, a target organ or tissue of interest is or comprises bone marrow, breast, ovary, coronary artery, fat, muscle, skin (e.g., epidermis, dermis, subcutis, etc.), lymph node, heart, spleen, lung, kidney, (e.g., renal glomeruli), dura mater, adrenal gland, testis, prostate, bladder, brain (e.g., cerebellum, cerebrum), thyroid, aorta, esophagus, stomach, duodenum, pancreas (e.g., pancreatic islet), gall bladder, liver, large bowel, small bowel, stem cells, stromal cells, endothelial cells, or combinations thereof. In some embodiments, a target organ or tissue of interest is or comprises at least one, at least two, at least three, at least four, at least five, at least six, or more of bone marrow, breast, ovary, coronary artery, fat, muscle, skin (e.g., epidermis, dermis, subcutis, etc.), lymph node, heart, spleen, lung, kidney, (e.g., renal glomeruli), dura mater, adrenal gland, testis, prostate, bladder, brain (e.g., cerebellum, cerebrum), thyroid, aorta, esophagus, stomach, duodenum, pancreas (e.g., pancreatic islet), gall bladder, liver, large bowel, small bowel, stem cells, stromal cells, and endothelial cells.

Various sample processing steps may be accomplished to prepare a sample for quantification. In some cases, crude sample (e.g., whole tissue, homogenized tissue, etc.) will suffice. In some cases, highly purified template for quantification is preferred.

### Quantification Methodologies

Quantification methodologies of the present disclosure make use of a sample which includes a nucleic acid template for amplification. The nucleic acid template may be of any type, e.g., genomic DNA, RNA, plasmids, bacteriophages, and/or artificial sequences. The nucleic acid template may be from any source, e.g., whole organisms, organs, tissues, cells, organelles (e.g., chloroplasts, mitochondria), synthetic nucleic acid sources, etc.

Conventional phage display methodologies typically utilize quantitation of transducing units (TU-counting) of phage recovered from a sample. Generally, phage particles are recovered by infection and amplification in host bacteria growing in medium under either a selective genetic pressure (such as antibiotic resistance) or a differential identifying color scheme. Host bacteria allow viral multiplication and generate thousands of newly-formed phage particles. Upon plating of the host bacteria, lysogenic phage (i.e., non-lytic M13-derived) yields bacterial colonies whereas lytic phage (i.e., Lambda-derived) generates plaques in a bacterial lawn; each resulting bacterial colony or phage plaque is considered a transducing-unit (TU).

The present disclosure encompasses the recognition that real-time PCR allows rapid quantification of phage display libraries recovered from a sample, in some cases in the absence of bacteria. Real-time PCR provides an accurate and sensitive means of quantifying DNA in a sample. General principles of real time quantitative PCR are known in the art. Typically, real time quantitative PCR procedures follow the general pattern of PCR, but the amplified DNA is quantified during each cycle. Two common methods of quantification are the use of fluorescent dyes that intercalate with double-stranded DNA and modified DNA oligonucleotide primers or probes the fluorescence of which changes during one of the steps of the PCR. Generally, encoded ligands are expressed from a vector containing a primer recognition site useful for amplification of the insert or a portion thereof. For example, a common feature to most vectors derived from the fUSE5 vector have a TetR gene to which oligonucleotide primers may be designed. Use of a common primer recognition site in a phage library allows amplification of multiple sequences from two or more phage within a library using a single set of PCR primers. In some embodiments, sequences from two or more phage with a library are amplified simultaneously.

Real time PCR using dyes which bind to double-stranded DNA can be used to quantitate phage in accordance with the present disclosure. For example, a DNA-binding dye, such as SYBR Green, binds to all double-stranded (ds)DNA in a PCR reaction, causing increased fluorescence of the dye. An increase in DNA product during PCR therefore leads to an increase in fluorescence intensity which is measured at each cycle, thus allowing DNA concentrations to be quantified.

In some embodiments, a reporter probe is used in real time PCR assays in accordance with the present disclosure. For example, in a TAQMAN™ (a trademark of Roche Molecular Systems) real-time PCR assay, a quenched fluorescent probe allows quantitation of amplified nucleic acids in real time. (See, e.g., Heid et al. (1996) “Real time quantitive PCR,” Genome Research. 6:986-994 and Gibson et al. (1996) “A novel method for real time quantitative RT-PCR,” Genome Research. 6:995-1001, the entire contents of both of which are herein incorporated by reference.) The quenched fluorescent probe typically comprises an oligonucleotide designed to hybridize to a nucleic acid, typically a PCR amplification product of interest (e.g., an amplicon from a target locus or reference locus) conjugated to a fluorophore and to a fluorescent quencher. The fluorescent quencher is normally in proximity to the fluorophore on a given TAQMAN™; therefore, no signal can be detected from the fluorophore. When a TAQMAN™ probe molecule is hybridized to a nucleic acid that is being amplified, the fluorophore can be released from the probe by exonuclease activity of the polymerase during the extension portion of an amplification cycle. Once released from the probe and (thus away from the quencher), a fluorophore can be detected. When excited by the appropriate wavelength, the fluorophore will emit light of a particular wavelength spectrum characteristic of that fluorophore. Detectable signal from the fluorophore can therefore be indicative of amplification product. As fluorescent signal in a sample can be measured in real time, TAQMAN™ real time PCR allows quantitation of amplification product in real time, e.g., at each amplification cycle.

Methods of determining a standard curve (e.g., by linear regression analysis) and/or determining nucleic acid concentration are known in the art and can be considered routine.

### Characterization of Ligands

Any of a variety of well-known, conventional methods can be used to sequence the DNA molecules isolated by a method of the invention. In some embodiments, sequencing DNA molecules isolated by a method of the invention includes use of fluorescence. In some embodiments, sequencing DNA molecules isolated by a method of the invention does not include fluorescence. In some embodiments, methods of the present disclosure can be adapted for sequencing with any high throughput sequencing method. Typical such methods which are described herein include, but are not limited to, the sequencing technology and analytical instrumentation offered by Roche 454 Life Sciences™, Branford, Conn., which is sometimes referred to herein as “454 technology” or “454 sequencing.”; the sequencing technology and analytical instrumentation offered by Illumina, Inc, San Diego, Calif. (their Solexa Sequencing technology is sometimes referred to herein as the “Solexa method” or “Solexa technology”); the sequencing technology and analytical instrumentation offered by ABI, Applied Biosystems, Indianapolis, Ind., which is sometimes referred to herein as the ABI-SOLiD™ platform or methodology; or the sequencing technology and analytical instrumentation offered by Ion Torrent™, San Fransisco, Calif., among others.

One sequencing method that can be used on nucleic acid molecules isolated by methods of the present disclosure is the 454 method. This method uses a 454 Genome Sequencer 20 or FLX (454 Life Sciences, Roche Applied Sciences). See, e.g., Margulies et al. (2005) Nature 437, 376-80; Rogers et al. (2005) Nature 437, 326-7; or the technical manual available on the web site for 454 Life Sciences. See also the patent application assigned to the 454 company, US2005/0079510. Such devices have extremely high throughput. Generally, between about 80 and about 130 bases are sequenced with the Genome Sequencer 20 apparatus, or between about 200 and 250 bases with the FLX apparatus. An accurate read of about 100 bases is currently claimed by the 454 Life Sciences company for the Genome Sequencer 20 apparatus, and an accurate read of about 230 is claimed by the current version of the machine, the FLX apparatus. Suitable reagents for carrying out the sequence reactions can be purchased from commercial suppliers, such as Roche Applied Biosciences (Indianapolis, Ind.).

Another sequencing method that can be employed is a the conventional Solexa Sequencing technology (offered by Illumina). Sequencing with this device involves bridge amplification on a solid surface, as described, e.g., on the web site for the Promega company and the web site for Illumina (Solexa). Bridge amplification employs primers bound to a solid surface for the extension and amplification of solution phase target nucleic acid sequences. The term “bridge amplification” refers to the fact that, during the annealing step, the extension product from one bound primer forms a bridge to the other bound primer. All amplified products are covalently bound to the surface. Because the Solexa sequencing method involves an A and a B primer, DNA molecules ligated to adaptors A and B of the invention can also be sequenced by this method. Conventional procedures for using this apparatus are well known in the art, and are available from the manufacturer. In general, sequencing with the Solexa sequencing method is not directional, so portions of both ends of a DNA molecule of interest are generally sequenced. The method may be adapted to allow sequencing from one end of particular interest.

Another sequencing method that can be used is a the conventional sequencing method utilizing a the Applied Biosystems SOLiD™ sequence technology (from Roche Applied Biosciences, Indianapolis, Ind.). The Applied Biosystems SOLiD™ System is a genetic analysis platform that enables massively parallel sequencing of clonally amplified DNA fragments linked to magnetic beads. The sequencing methodology is based on sequential ligation with dye-labeled oligonucleotides. In this method, the DNA sequence is generated by measuring the serial ligation of an oligonucleotide by ligase. All fluorescently labeled oligonucleotide probes are present simultaneously and compete for incorporation. After each ligation, the fluorescence signal is measured and then cleaved before another round of ligation takes place.

Yet another sequencing method that can be used in accordance with the present disclosure is semiconductor sequencing, such as for example, methodologies and systems provided by Ion Torrent™ (San Fransisco, Calif.). The Ion Torrent™ system utilizes arrays of chemical-sensitive field effect transistors to detect incorporation of nucleotides into growing strands of DNA by measuring changes in current. In nature, when a nucleotide is incorporated into a strand of DNA by a polymerase, a hydrogen ion is released as a biproduct. Semiconductor sequence technology, such as Ion Torrent™, directly measures the pH change caused by release of a positively-charged hydrogen ion. Semiconductor sequence technology does not rely on incorporation or detection of fluorescent molecules or signals.

DNA sequence data may be filtered or analyzed by any available method. For example, in some embodiments, DNA sequence data is filtered to eliminate singleton sequences.

### Use of Ligands

Ligands identified in accordance with the present disclosure can be useful for directing to a selected organ or tissue a therapeutic agent, diagnostic agent or imaging agent, a tag or insoluble support, a liposome or a microcapsule comprising, for example, a permeable or semipermeable membrane, wherein an agent such as a drug to be delivered to a selected organ or tissue is contained within the liposome or microcapsule (for example, as described in U.S. Pat. No. 6,933,281, the entire contents of which is herein incorporated by reference). These and other moieties known in the art can be used in accordance with the present disclosure.

In some embodiments, a moiety can be a detectable agent such as a radionuclide or an imaging agent, which allows detection or visualization of the selected organ or tissue. Thus, the invention provides a conjugate comprising a ligand linked to a detectable agent. The type of detectable agent selected will depend upon the application. For example, for an in vivo diagnostic imaging study of the lung in a subject, a lung homing molecule can be linked to an agent that, upon administration to the subject, is detectable external to the subject. For detection of such internal organs or tissues, for example, the prostate, a gamma ray emitting radionuclide such as indium-113, indium-115 or technetium-99 can be linked to a prostate homing molecule and, following administration to a subject, can be visualized using a solid scintillation detector. For organs or tissues at or near the external surface of a subject, for example, retina, a fluorescein-labeled retina homing molecule can be used such that the endothelial structure of the retina can be visualized using an opthalamoscope and the appropriate optical system.

Ligands that localize to a pathological lesion in an organ or tissue similarly can be linked to an appropriate detectable agent such that the size and distribution of the lesion can be visualized. For example, where a ligand localizes to a normal organ or tissue, but not to a pathological lesion in the organ or tissue, the presence of the pathological lesion can be detected by identifying an abnormal or atypical image of the organ or tissue, for example, the absence of the detectable agent in the region of the lesion.

A detectable agent also can be an agent that facilitates detection in vitro. For example, in some embodiments, a conjugate comprising a ligand linked to an enzyme produces a visible signal when an appropriate substrate is present. In some embodiments, a conjugate comprises alkaline phosphatase or luciferase. In some embodiments, a conjugate is used in immunohistochemistry methods. In some embodiments, a conjugate also is used to detect the presence of a target molecule to which a ligand binds in a sample.

In some embodiments, a moiety can be a therapeutic agent. Thus, the invention provides a conjugate comprising a ligand linked to a therapeutic agent. A therapeutic agent can be any biologically useful agent that, when linked to a ligand identified by methods provided by the present disclosure, exerts its function at the site of the selected organ or tissue. For example, a therapeutic agent can be a small organic molecule that, upon binding to a target cell due to the linked ligand, is internalized by the cell where it can effect its function. A therapeutic agent can be a nucleic acid molecule that encodes a protein involved in stimulating or inhibiting cell survival, cell proliferation or cell death, as desired, in the selected organ or tissue.

For example, in some embodiments, a therapeutic agent is an antisense nucleic acid molecule. In some embodiments, a therapeutic agent is an siRNA nucleic acid molecule. In some embodiments, nucleic acid molecules are modified oligonucleotides that are resistant to endogenous nucleases, e.g., exonucleases and/or endonucleases, and are therefore stable in vivo. Modifications, such as phosphorothioates, have been made to nucleic acids to increase their resistance to nuclease degradation, binding affinity and uptake. Exemplary nucleic acid molecules for use as antisense and/or siRNA oligonucleotides are phosphoramidate, phosphothioate and methylphosphonate analogs of DNA (see also U.S. Pat. Nos. 5,176,996; 5,264,564; and 5,256,775). Nucleic acids can be DNA, RNA, or chimeric mixtures or derivatives or modified versions thereof, single-stranded or double-stranded.

In some embodiments, a useful therapeutic agent that stimulates cell death is ricin, which, when linked to a ligand identified by provided methods, can be useful for treating a hyperproliferative disorder, for example, cancer. A conjugate comprising a ligand identified by provided methods and an antibiotic, such as ampicillin or an antiviral agent such as ribavirin, for example, can be useful for treating a bacterial or viral infection in a selected organ or tissue.

A therapeutic agent also can inhibit or promote the production or activity of a biological molecule, the expression or deficiency of which is associated with the pathology. Thus, in some embodiments, a protease inhibitor is a therapeutic agent that, when linked to a ligand, inhibits protease activity at the selected organ or tissue. A gene or functional equivalent thereof such as a cDNA, which can replenish or restore production of a protein in a selected organ or tissue, also can be a therapeutic agent useful for ameliorating the severity of a pathology. A therapeutic agent also can be an antisense nucleic acid molecule, the expression of which inhibits production of a deleterious protein, or can be a nucleic acid molecule encoding a dominant negative protein or a fragment thereof, which can inhibit the activity of a deleterious protein.

In some embodiments, the present disclosure provides a conjugate comprising a ligand linked to a tag. A tag can be, for example, an insoluble support such as a chromatography matrix, or a molecule such as biotin, hemagglutinin antigen, polyhistidine, T7 or other molecules known in the art. Such a conjugate comprising a tag can be useful to isolate a target molecule, to which the ligand binds.

When administered to a subject, a conjugate comprising a ligand and a moiety is administered as a pharmaceutical composition containing, for example, the conjugate and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters.

A pharmaceutically acceptable carrier can contain physiologically acceptable compounds that act, for example, to stabilize or to increase the absorption of the complex. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the composition. The pharmaceutical composition also can contain an agent such as a cancer therapeutic agent or other therapeutic agent as desired.

One skilled in the art would appreciate that a pharmaceutical composition containing ligands identified by provided methods can be administered to a subject by various routes including, for example, orally or parenterally, such as intravenously. The composition can be administered by injection or by intubation. The pharmaceutical composition also can be a ligand linked to a moiety such as a liposome or other polymer matrix, which can have incorporated therein, for example, a drug that promotes or inhibits cell death (Gregoriadis, Liposome Technology, Vol. 1 (CRC Press, Boca Raton, Fla. 1984), which is incorporated herein by reference). Liposomes, for example, which consist of phospholipids or other lipids, are nontoxic, physiologically acceptable and metabolizable carriers that are relatively simple to make and administer.

In performing a diagnostic or therapeutic method as disclosed herein, an effective amount of a conjugate comprising a ligand must be administered to the subject. An “effective amount” is the amount of the conjugate that produces a desired effect. An effective amount will depend, for example, on the moiety linked to the ligand and on the intended use. For example, a lesser amount of a radiolabeled ligand can be required for imaging as compared to the amount of the radiolabeled ligand administered for therapeutic purposes, where cell killing is desired. An effective amount of a particular conjugate for a specific purpose can be determined using methods well known to those in the art.

The route of administration of a ligand will depend, in part, on the chemical structure of the organ homing molecule. Peptides, for example, are not particularly useful when administered orally because they can be degraded in the digestive tract. However, methods for chemically modifying peptides to render them less susceptible to degradation by endogenous proteases or more absorbable through the alimentary tract are well known (see, for example, Blondelle et al., supra, 1995; Ecker and Crooke, supra, 1995; Goodman and Ro, supra, 1995). Such methods can be performed on peptides that home to a selected organ or tissue. In addition, methods for preparing libraries of peptide analogs such as peptides containing D-amino acids; peptidomimetics consisting of organic molecules that mimic the structure of a peptide; or peptoids such as vinylogous peptoids, have been previously described above and can be used to identify homing molecules suitable for oral administration to a subject.

In principle, ligands identified by provided methods can have an inherent biological property, such that administration of the ligand provides direct biological effect. For example, a ligand can be sufficiently similar to a naturally occurring ligand for the target molecule that the organ homing molecule mimics the activity of the natural ligand. Such a ligand can be useful as a therapeutic agent having the activity of the natural ligand. For example, where the ligand mimics the activity of a growth factor that binds a receptor expressed by the selected organ or tissue, such as a skin homing ligand that mimics the activity of epidermal growth factor, administration of the ligand can result in cell proliferation in the organ or tissue. Such inherent biological activity of an organ homing ligand of the invention can be identified by contacting the cells of the selected organ or tissue with the homing ligand and examining the cells for evidence of a biological effect, for example, cell proliferation or, where the inherent activity is a toxic effect, cell death.

In addition, ligands identified by provided methods can have an inherent activity of binding a particular target molecule such that a corresponding ligand cannot bind the receptor. It is known, for example, that various types of cancer cells metastasize to specific organs or tissues, indicating that the cancer cells express a ligand that binds a target molecule in the organ to which it metastasizes. Thus, administration of a lung homing ligand, for example, to a subject having a tumor that metastasizes to lung, can provide a means to prevent the potentially metastatic cancer cell from becoming established in the lung. In general, however, ligands identified by provided methods are useful for targeting a moiety to a selected organ or tissue, including, but not limited to lung, skin, pancreas, retina, prostate, ovary, lymph node, adrenal gland, liver or gut. Thus, the present disclosure provides methods of treating a pathology in a selected organ or tissue by administering to a subject having the pathology a conjugate comprising ligands identified by provided methods linked to a therapeutic agent.

The following examples are intended to illustrate but not limit the present invention.

### EXEMPLIFICATION

### Example 1

### Phage Display System with Improved Quantification and Ligand Analysis

The present Example describes phage display library screening and demonstrates, among other things, that the use of real-time PCR allows rapid quantification of phage in order to enable large scale quantification in the absence of bacterial amplification of phage.

### Materials & Methods

### Ethics Statement

This study design was reviewed and approved by the Institutional Review Board of the University of Texas M. D. Anderson Cancer Center and followed a pre-established ethics framework [18]-[19].

### Phage Preparation

Insertless Fd-tet [5] or RGD-4C phage [6]-[10] were amplified overnight (ON) at 37° C. from a single MC1061 E. coli as described [1]. Phage particles were precipitated with ice-cold phosphate-buffered saline (PBS) containing 15% NaCl and PEG 8000 for 1 hour, centrifuged for 20 minutes at 4° C. at 10,400 g, re-suspended in 5 ml sterile PBS, and again precipitated in ice-cold PBS containing 15% NaCl and PEG 8000 for 30 minutes. The final pellet was re-suspended in 100 μl sterile PBS and centrifuged for 2 minutes at maximum g force, and the supernatant was sterile filtered.

### Cell Binding and Internalization Assays

Bone marrow-derived endothelial cells [33] were incubated (105 cells in 100 μl) with Fd-tet or RGD-4C phage (109, 3×109, and 1×1010 TU) in ice-cold DMEM containing 3% BSA for 3 hours. Cell suspensions were placed in Eppendorf tubes and centrifuged at 10,000 g for 10 minutes through an aqueous-organic interface [9], [16], [17], [29]. Cell pellets carrying membrane-bound phage were resuspended in 100 μl of PBS. Phage titers were quantified by TU-counting or qPhage.

Cells were seeded onto 12-well plates (105 cells/well) ON, blocked with Dulbecco's modified Eagle's medium (DMEM) containing 30% fetal bovine serum (FBS) at 37° C. and incubated with 4×108 TU of phage in DMEM containing 2% FBS (400 μl of a 2×109 TU/ml solution). Internalized phage were detected with a rabbit anti-bacteriophage antibody (Sigma) and Cy3-conjugated anti-rabbit antibody or were quantified by qPhage after DNA extraction.

### Patient Tissue Collection

After surrogate written informed consent was obtained from the legal next of kin [18], [19], [20], short-term intravenous infusion of phage was performed as described [20] followed by representative tissue biopsies of skin, fat-tissue, bone marrow and skeletal-muscle. Besides the screening reported here, which derived from biopsies taken from a single individual, the library was previously administered in another two patients [20] using the synchronous selection methodology [29]. Biopsy samples served for simultaneous histopathology analyses, host bacterial infection, qPhage, and next-generation sequencing.

DNA Extraction and qPhage

DNA extractions were performed with DNeasy (Qiagen). Phage content was determined by quantitative PCR (qPCR). PCR templates consisted of 5 μl of a 1:20 dilution of DNA, 1× Power SYBR Green PCR Master Mix (Applied Biosystems) and 3.75 pmol of each oligonucleotide primer (fUSE5F1: 5′-TGAGGTGGTATCGGCAATGA-3′ and fUSE5R1: 5′-GGATGCTGTATTTAGGCCGTTT-3′) directed to the amplification of a fragment of the TetR gene, in a final reaction volume of 15 μl. The program consisted of 50° C. for 2 minutes, 95° C. for 10 minutes, followed by 40 amplification cycles of 95° C. for 15 seconds and 60° C. for 1 minute. Standard curves were generated with serial phage dilutions (from 3 to 38 plasmids) for each run. Each point of the curve and each sample DNA were amplified in triplicates. The standard curve was calculated by a linear regression analysis and serial dilutions. Amplification efficiency (AE) of each PCR cycle was calculated from the slope (s) of the standard curve by the equation: AE=101/(−s).

### Phage DNA Amplification for Next-Generation DNA Sequencing

The amplification of the insert-containing region in the pIII gene was performed with the oligonucleotide set fUSE5454F: 5′-CGCAATTCCTTTAGTTGTTCC-3′ and fUSE5454R: 5′-TGAATTTTCTGTATGAGGTTTTGC-3′. The reaction mix consisted of 6 pmol of each primer, and 12.5 μl of the 2× Phusion hot-start high-fidelity DNA polymerase mix (Finnzymes) in 25 μl final volume. Amplifications were performed with a high-fidelity DNA polymerase mix. Cycles varied from 20-25, and the number of reaction tubes varied from 2-6, according to the amount of phage available in each sample. Resulting amplicons (12-18 μg) served for adaptor ligation and next-generation sequencing with the FLX platform (Roche/454). All sequences described here are presented in a Supporting Information file accompanying this paper, available on line.

### Bioinformatics

DNA sequencing data were filtered to keep only sequences expected from a 21-nt inserts (termed NNB) of a CX7C library. Due to the much larger-scale nature of our sequencing approach, we adopted stringent criteria for accepting DNA sequencing reads; we applied a singleton-elimination filter where sequences that appeared only once in the whole dataset were not considered. This filter cannot be used in datasets derived from Sanger-sequencing, due to their relatively small size, or in non-selected CX7C library datasets, where only singletons are expected. Tests were performed to compare the similarity of Sanger- and next-generation sequencing-derived datasets in terms of peptide composition, GC and homopolymer content, codon usage, and residue frequency. Saturation plots were obtained by randomly shuffling the order of the filtered nucleotide or peptide sequences, and by calculating the number of distinct accumulated sequences at every 30th sequence. We defined coverage as the percent ratio between the number of observed distinct peptides and the total number of predicted distinct peptides in each tissue as estimated with EstimateS [34]. Three estimators were used: abundance-based coverage estimator (ACE), incidence-based coverage estimator (ICE), and CHAO 1 mean [35]. The adopted total number of predicted distinct peptides per sample was the average of these three estimates. Data processing scripts were written in Perl v5.8 and analyses were done with The R Project for Statistical Computing R-2.6.2.

### Results

### DNA-Based Analysis of Phage Quantification

The present disclosure encompasses the recognition that development of a high-throughput platform for phage display selection that is “bacteria-free” (e.g., quantification, internalization, and determination of the insert sequence that is largely based on direct nucleic acid analysis) may be faster and more cost-effective than the current methodology. To validate the platform described herein, all intermediate steps from phage quantification to sequence determination were evaluated in a direct, simultaneous (i.e., side-by-side) comparison with conventional phage display methodology.

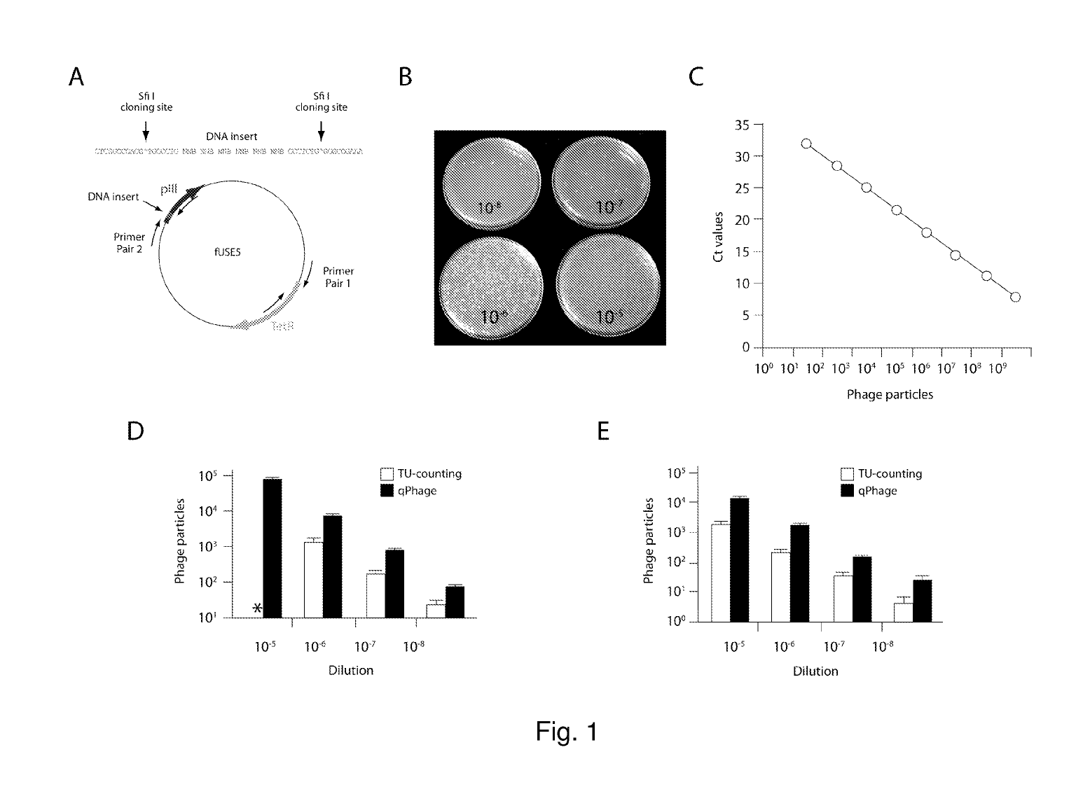

We started by comparing the phage quantification range obtained through conventional TU-counting versus a quantitative real-time PCR-based approach (termed “qPhage”). For the qPhage methodology, we designed oligonucleotides targeting the TetR gene, a common feature to most vectors derived from fUSE5 (FIG. 1A). Both methods (TU-counting and qPhage) were used to determine phage particle content and titers in multiple serial dilutions from defined stocks. During conventional TU counting, high bacterial colony density prevented accurate determination of particles at the lower plating dilutions (FIG. 1B), a result mimicking experiments in vivo when high concentrations of phage are found in a targeted tissue. In contrast, the qPhage method consistently detected and linearly quantified phage content in the range of dilutions evaluated (FIG. 1C). The results of several (n=7) independent amplifications of such phage dilutions showed a mean correlation coefficient of 0.998±0.002 and a mean amplification slope of −3.42±0.06, an amplification efficiency of ˜96%. The direct comparison of both methods, by the use of the same preparation of 4 points of 10-fold serial dilutions of an insertless phage [5] and a phage displaying an RGD-4C peptide targeting αv integrins [6]-[9], showed that qPhage is at least 5-fold more sensitive than the conventional TU-counting. This increased sensitivity is derived from the capability of the DNA-based method to quantify non-infective phage particles. When the same serial dilutions of RGD-4C phage or Fd-tet phage (FIGS. 1D and 1E) were either plated or quantified by qPhage, each 10-fold dilution point resulted on average in a proportional variation of 7-fold for TU-counting and 9.3-fold for qPhage, with an SEM of the triplicates at all dilutions of 32.5% for TU-counting, versus 7.8% for qPhage. This direct comparison allowed an evaluation of both approaches in the detection of pre-determined amounts of phage particles, as well as measurement of their detection limits and linear quantification ranges. Based on these direct comparative results, we conclude that the qPhage methodology is superior and less human error-prone, particularly in high-density colony plating settings and under experimental conditions in which suitable dilutions for TU-counting and relative quantification are difficult to predict.

Phage Binding and Internalization into Cells

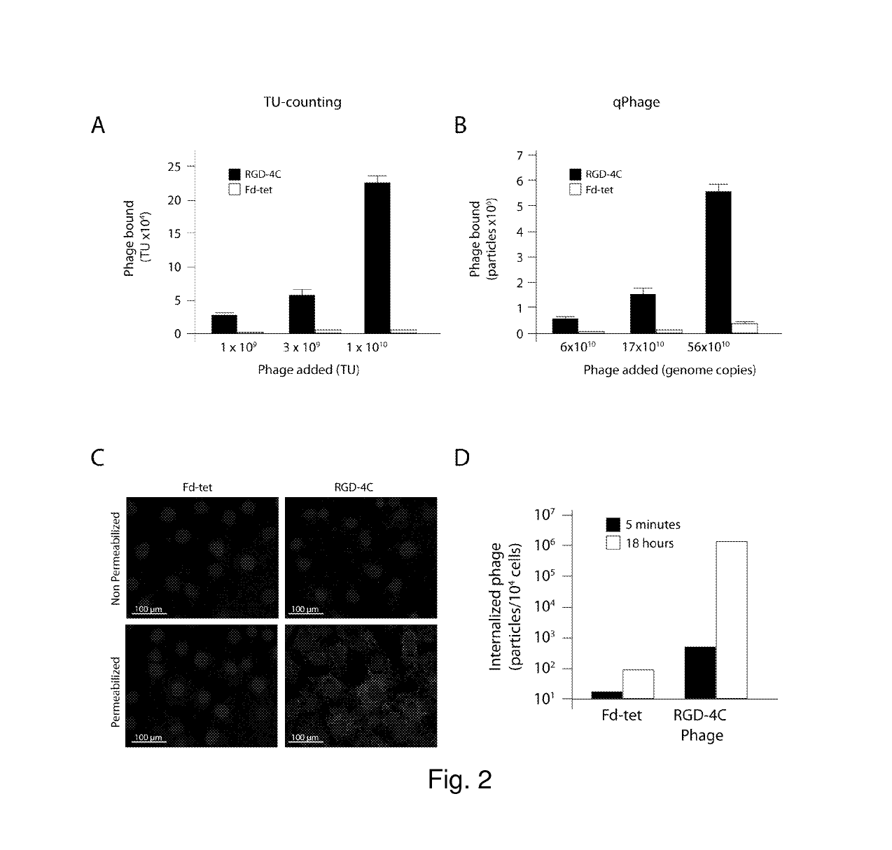

Having shown that the qPhage methodology is better than conventional TU-counting for quantification, we next sought to compare these techniques in phage display selection assays [9]-[17]. Binding of RGD-4C phage [6]-[10] or insertless control Fd-tet phage [5] to endothelial cells was performed by the Biopanning and Rapid Analysis of Selective Interactive Ligands (BRASIL) method [9] and cell-bound phage was quantified concomitantly with either TU-counting (FIG. 2A) or qPhage (FIG. 2B). Good correlation between both methods was observed, indicating that qPhage recapitulates a state-of-the-art methodology for phage-cell binding.

Another application of qPhage was the precise quantification of phage uptake in mammalian cells, thereby allowing analysis of its internalization dynamics. In the conventional methodology, phage internalization is achieved only after prolonged incubation, defined here as an overnight (ON), of phage and cells as detected by cell membrane permeabilization and visualization with anti-phage antibodies (FIG. 2C). Given the inherent technical challenges of staining-based immunodetection, this approach allows one to determine only whether or not a certain targeting peptide mediates cell internalization of a phage particle in a non-quantitative or at best, semi-quantitative manner. In contrast, an accurate quantification of phage internalization was obtained with the qPhage method. Traces of phage internalization, even after very short incubation intervals (<10 min), were detectable by qPhage but not by immunofluorescence (FIG. 2D). These results show that the targeted RGD-4C phage internalizes far more than the Fd-tet phage. Even after prolonged incubation, internalization of RGD-4C phage was 104-fold higher than that of Fd-tet phage for the same period of time. The detection of internalized RGD-4C phage after the short incubation interval used was not as dramatic as that seen after ON incubation, but still points to an uptake ˜10-fold higher than that observed with insertless phage, which suggests rapid cell internalization of RGD-4C. These results indicate that internalization of peptide-guided phage can be finely quantified by qPhage. The DNA-based qPhage methodology allows full quantification of phage content, binding, and cell internalization, with a superior performance compared to conventional TU-counting.

### Next-Generation Phage Amplicon Deep Sequencing

To address the other rate-limiting step of conventional phage display, we reasoned that this DNA-based system would have to allow determination of the sequences of the encoded ligands (targeting peptides, unless otherwise specified) in large-scale. We adapted the next-generation pyrosequencing methodology (454/Roche) for phage DNA; for proof-of-principle, we used tissues obtained after intravenous administration of a phage library to end-of-life patients [18]-[20]. To determine the capacity of this approach for the high-throughput generation of phage sequences, we produced insert-containing amplicons from surgical biopsies of four human tissues (skin, white adipose tissue, bone marrow, and skeletal muscle), with oligonucleotides flanking the DNA insert coding for the peptide in the pIII gene (FIG. 1A).

In a single next-generation run, we generated a total of 319,361 sequences: 251,032 derived from tissues plus another 68,329 derived from the unselected CX7C library before administration. After filtering (see Methods), the dataset was compared to 3,840 sequences derived from the same samples by conventional Sanger-sequencing of phage recovered from host bacteria (Table 1).

TABLE 1
Sequence datasets derived from conventional and DNA-based phage display
selection.

| | TU-counting & Sanger Sequencing | | | | |
|---|---|---|---|---|---|
| | | Sequences | | | |
| | | remaining | Sequences remaining | | |
| | | after eliminating | after eliminating | | |
| | Total raw | inserts ≠ 21 | non-NNB inserts | Distinct | Distinct peptide |
| Sample | sequences | nt (%) | (final dataset) | nucleotide sequences | sequences |
| Bone | 1056 (100) | 979 (92.7) | 953 (90.2) | 555 | 553 |
| marrow | | | | | |
| Fat | 672 (100) | 648 (96.4) | 625 (93) | 357 | 356 |
| Muscle | 1056 (100) | 1008 (95.5) | 947 (92.2) | 522 | 522 |
| Skin | 1056 (100) | 1029 (97.4) | 1002 (94.9) | 496 | 496 |
| CX7C | N/A | N/A | N/A | N/A | N/A |
| library | | | | | |
| TOTAL** | 3840 (100) | 3664 (95.4) | 3554 (92.6) | 1289 | 1285 |

| | qPhage & Next-generation sequencing | | | | | |
|---|---|---|---|---|---|---|
| | | | | Sequences | | |
| | | Sequences | Sequences | remaining | | |
| | | remaining | remaining | after | | |
| | | after | after | eliminating | | |
| | | eliminating | eliminating | singletons | Distinct | Distinct |
| | Total raw | inserts ≠ 21 | non-NNB | (final | nucleotide | peptide |
| Sample | sequences | nt (%) | inserts | dataset) | sequences | sequences |
| Bone | 55,350 (100) | 39,847 (72) | 37,421 (67.6) | 37,049 (66.9) | 2541 | 2539 |
| marrow | | | | | | |
| Fat | 56,543 (100) | 46,356 (82) | 43,548 (77) | 43,439 (76.8) | 460 | 459 |
| Muscle | 78,157 (100) | 65,512 (83.8) | 61,005 (81.2) | 60,872 (77.9) | 1014 | 1011 |
| Skin | 60,982 (100) | 49,332 (80.9) | 46,106 (75.7) | 46,032 (75.5) | 979 | 976 |
| CX7C | 68,329 (100) | 57,089 (83.6) | 49,252 (72.1) | 49,525* (72.1) | 44,618 | 44,606 |
| library | | | | | | |
| TOTAL** | 319,361 (100) | 258,163 (80.8) | 237,392 (74.3) | | | |

*The final dataset for the CX7C reads includes singletons.
**Not including sequences from the non-injected CX7C library for TU counting.
doi:10.1371/journal.pone.0008338.t001

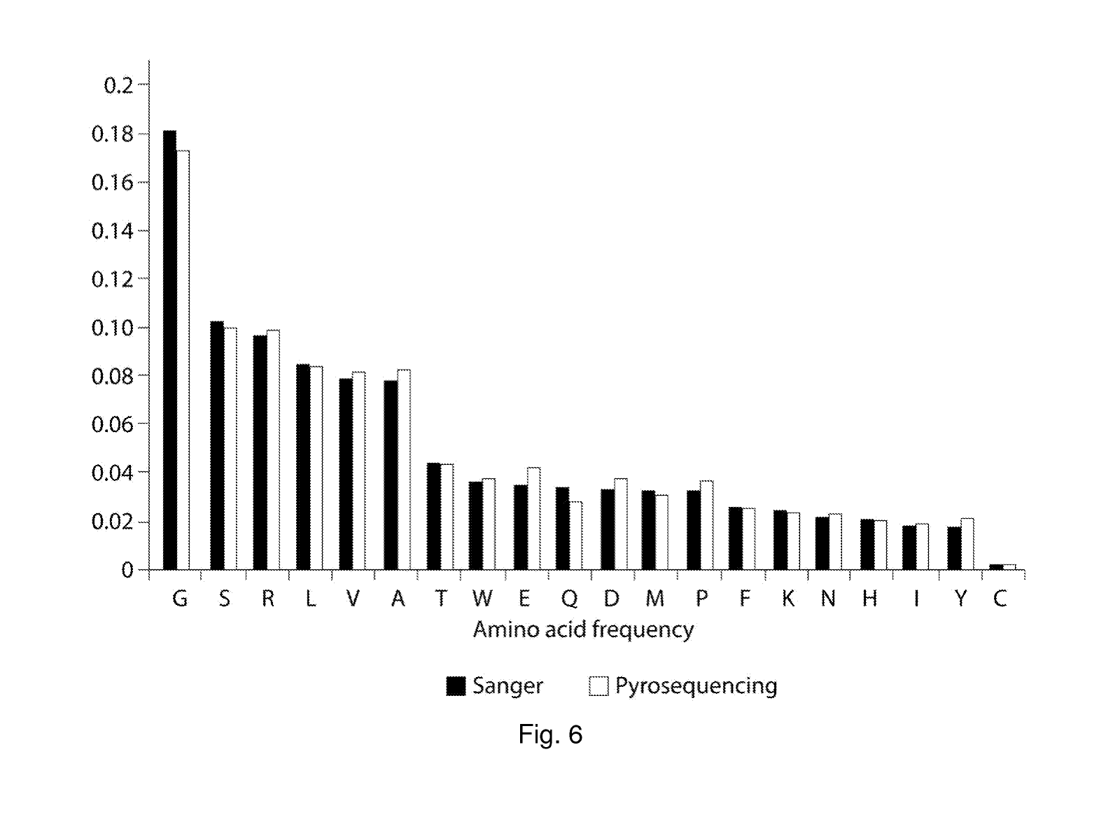

Our initial concern was to evaluate and, if possible, rule out eventual discrepancies caused by PCR amplification as a previous step to phage amplicon deep sequencing. Thus, quality-control and quality-assurance approaches were undertaken to compare the DNA sequences derived from both methodologies, including GC content, codon-usage, and amino acid frequencies in the encoded peptides as well as the overlap of actual peptides observed by the two methods and the frequency of homopolymers in inserts derived from each dataset. After evaluating a non-redundant set of 10,983 nucleotides from Sanger sequences and 55,587 nucleotides from the pyrosequencing platform, we observed no significant differences between the two approaches, which showed respective GC contents of 60.11% and 60.19% (2-sample, unequal variance T test; t-score=0.437, p-value ˜0.3). Strong correlations were observed for amino acid frequencies and codon utilization when 3,668 (Sanger-derived) or 18,445 (next-generation-derived) amino acid residues were compared (Pearson correlation analysis coefficients (r=0.996, p<0.001 and r=0.999, p<0.001, respectively), reinforcing the absence of nucleotide representation biases from DNA amplified phage (FIG. 6 and Table 2).

TABLE 2
Codon usage: TU-counting versus pyrosequencing.

| | | Next-generation | | | |
|---|---|---|---|---|---|
| | | Pyrosequencing | | Colony-counting | |
| Amino | Amino | | Percentage | | Percentage |
| Acid | Acid | Codon | (%) | Codon | (%) |
| A | A | GCG | 60.3 | GCG | 57.9 |
| A | A | GCT | 32.8 | GCT | 35.9 |
| A | A | GCC | 6.9 | GCC | 6.2 |
| C | C | TGT | 99.5 | TGT | 100.0 |
| C | C | TGC | 0.5 | TGC | 0 |
| D | D | GAT | 83.7 | GAT | 87.2 |
| D | D | GAC | 16.3 | GAC | 12.8 |
| E | E | GAG | 100.0 | GAG | 100.0 |
| F | F | TTT | 81.9 | TTT | 87.3 |
| F | F | TTC | 18.1 | TTC | 12.7 |
| G | G | GGG | 55.5 | GGG | 56.0 |
| G | G | GGT | 35.9 | GGT | 37.8 |
| G | G | GGC | 8.6 | GGC | 6.2 |
| H | H | CAT | 84.1 | CAT | 88.2 |
| H | H | CAC | 15.9 | CAC | 11.8 |
| I | I | ATT | 83.7 | ATT | 88.8 |
| I | I | ATC | 16.3 | ATC | 11.2 |
| K | K | AAG | 100.0 | AAG | 100.0 |
| L | L | TTG | 43.8 | TTG | 42.8 |
| L | L | CTG | 29.8 | CTG | 32.2 |
| L | L | CTT | 20.8 | CTT | 19.9 |
| L | L | CTC | 5.6 | CTC | 5.1 |
| M | M | ATG | 100.0 | ATG | 100.0 |
| N | N | AAT | 84.9 | AAT | 91.7 |
| N | N | AAC | 15.1 | AAC | 8.3 |
| P | P | CCG | 54.4 | CCG | 55.0 |
| P | P | CCT | 37.3 | CCT | 38.3 |
| P | P | CCC | 8.3 | CCC | 6.7 |
| Q | Q | CAG | 100.0 | CAG | 100.0 |
| R | R | CGG | 35.9 | CGG | 36.5 |
| R | R | AGG | 34.7 | AGG | 36.0 |
| R | R | CGT | 23.9 | CGT | 23.6 |
| R | R | CGC | 5.5 | CGC | 3.9 |
| S | S | TCG | 33.7 | AGT | 33.4 |
| S | S | AGT | 32.7 | TCG | 32.5 |
| S | S | TCT | 22.1 | TCT | 23.6 |
| S | S | AGC | 6.6 | AGC | 6.2 |
| S | S | TCC | 4.8 | TCC | 4.3 |
| T | T | ACG | 55.9 | ACG | 56.2 |
| T | T | ACT | 37.8 | ACT | 38.3 |
| T | T | ACC | 6.3 | ACC | 5.5 |
| V | V | GTG | 54.7 | GTG | 55.3 |
| V | V | GTT | 37.9 | GTT | 39.4 |
| V | V | GTC | 7.4 | GTC | 5.3 |
| W | W | TGG | 100.0 | TGG | 100.0 |
| Y | Y | TAT | 85.7 | TAT | 87.4 |
| Y | Y | TAC | 14.3 | TAC | 12.6 |

In Table 2, the codon usage frequency between both approaches was evaluated by applying a chi-square test, which indicated a significant association between both methods (p=0.004). Pearson correlation analysis indicated a strong positive correlation between pyrosequencing and colony-counting derived sequences (r=0.999, p<0.001).

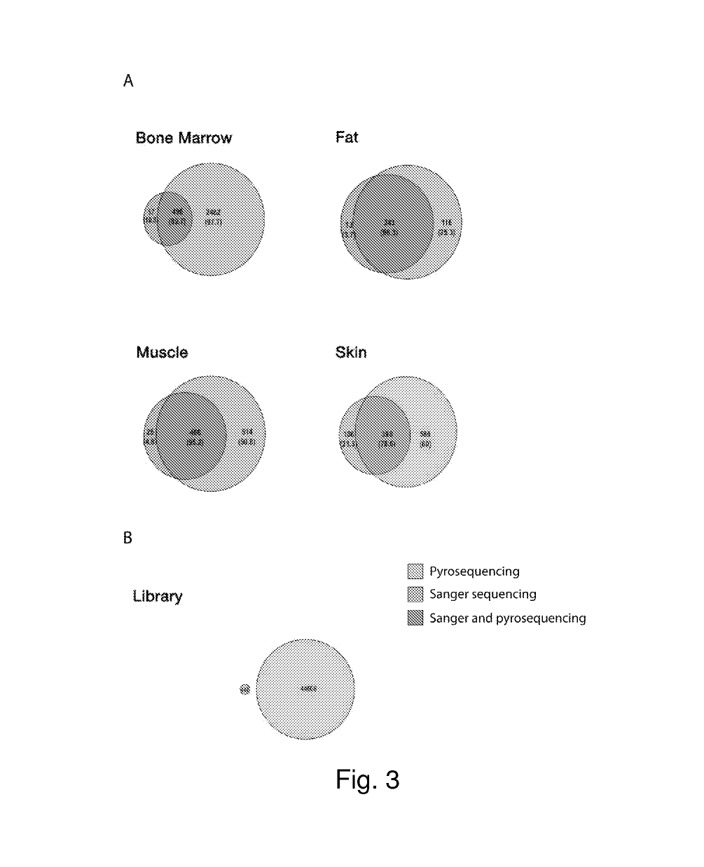

We conclude that there are no significant differences between phage DNA sequences recovered by the conventional methodology or by this alternative DNA-based approach. Moreover, when DNAs encoding peptide datasets derived from both methods were compared among human tissues (FIG. 3A) and the non-selected library (FIG. 3B), we observed that large-scale sequencing uncovered between 78.6 and 96.3% of the peptides revealed by TU-counting; whereas between 3.7 and 21.3% of the TU-derived sequences were not seen by next-generation sequencing, between 25.3 and 97.7% of the peptides revealed by deep sequencing could not be detected by TU-counting. Finally, no overlaps were seen when the non-selected phage library was sequenced by either method, a result indicating the absence of dominant clones prior to its administration (FIG. 3B).

As the next-generation DNA pyrosequencing methodology used here is known for its relatively high error-rate in DNA fragments containing homopolymers ≧5 nt [21]-[23] we performed a careful evaluation of this technical issue by comparing both sequence sets (i.e., Sanger-based versus 454 pyrosquencing-based). Due to the determined length of the peptide inserts (21nt), false-positives due to sequencing errors are not expected. However, a fraction of the rejected sequences may be false-negatives (i.e., phage containing valid sequences discarded due to a wrongly assigned non-21nt insert) derived from homopolymer sequencing errors. To determine the extent of this potential effect, we investigated the frequency of homopolymer-containing sequences in both, Sanger- and 454 pyrosequencing sets. When the accepted sequence sets derived from both methods were compared (Table 3), we observed that the frequency of homopolymer-containing sequences to be only slightly higher (and non-statistically significant, Chi-square test; P-value=0.9955) in the Sanger-dataset (20.4%) than in the pyrosequencing dataset (18.6%). If one assumes the percentage of 5 nt-homopolymer sequences from the Sanger-set (20.4%) to be true and takes into account the known 15% error rate of the pyrosequencing platform [22] one reaches an expected error rate of ˜3% (0.204×0.15) which is close to the 1.8% difference observed in the homopolymer frequency between datasets. Thus, together with other analyses presented herein, we conclude that the bias against homopolymer-containing inserts is small and non-significant in our dataset.

TABLE 3
Homopolymer-containing inserts in the accepted sequence datasets*

| Homopolymer | Accepted sequences | |
|---|---|---|
| size (nt) | 454-Pyrosequencing set | Sanger-sequencing set |
| 4 | 813/2847 (28.6%) | 371/1289 (28.8%) |
| 5 | 337/2847 (11.8%) | 159/1289 (12.3%) |
| 6 | 128/2847 (4.5%) | 65/1289 (5.0%) |
| 7 | 49/2847 (1.7%) | 24/1289 (1.9%) |
| 8 | 16/2847 (0.6%) | 15/1289 (1.1%) |
| ≧4 | 1343/2847 (47.2%) | 634/1289 (49.2%) |
| ≧5 | 530/2847 (18.6%) | 263/1289 (20.4%) |

(*Chi-square test, P = 20.9955).

### Phage Diversity and Motif Enrichment

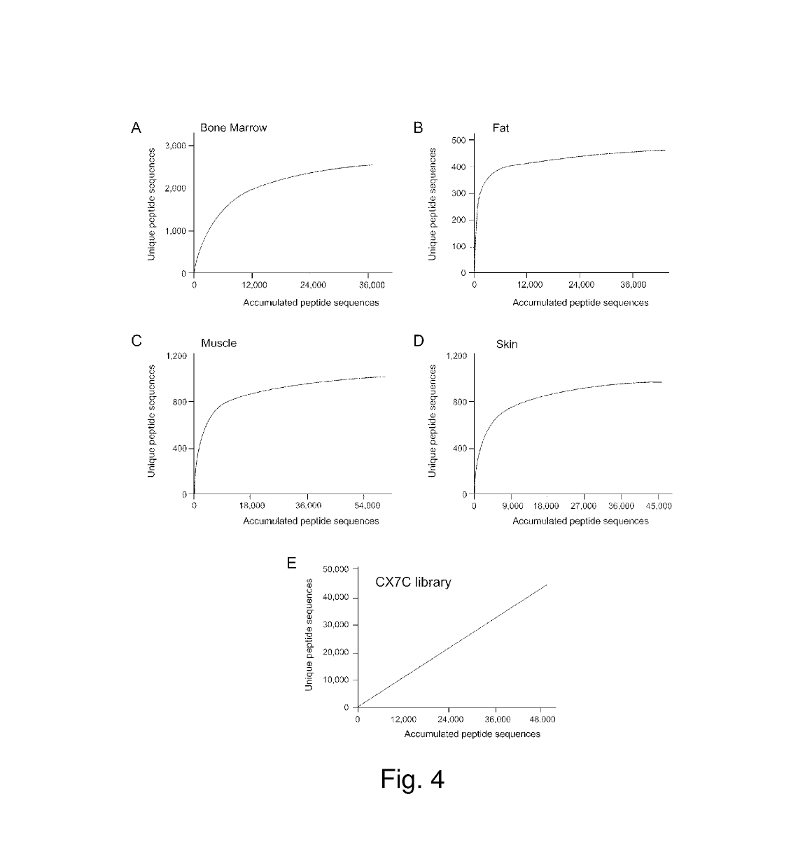

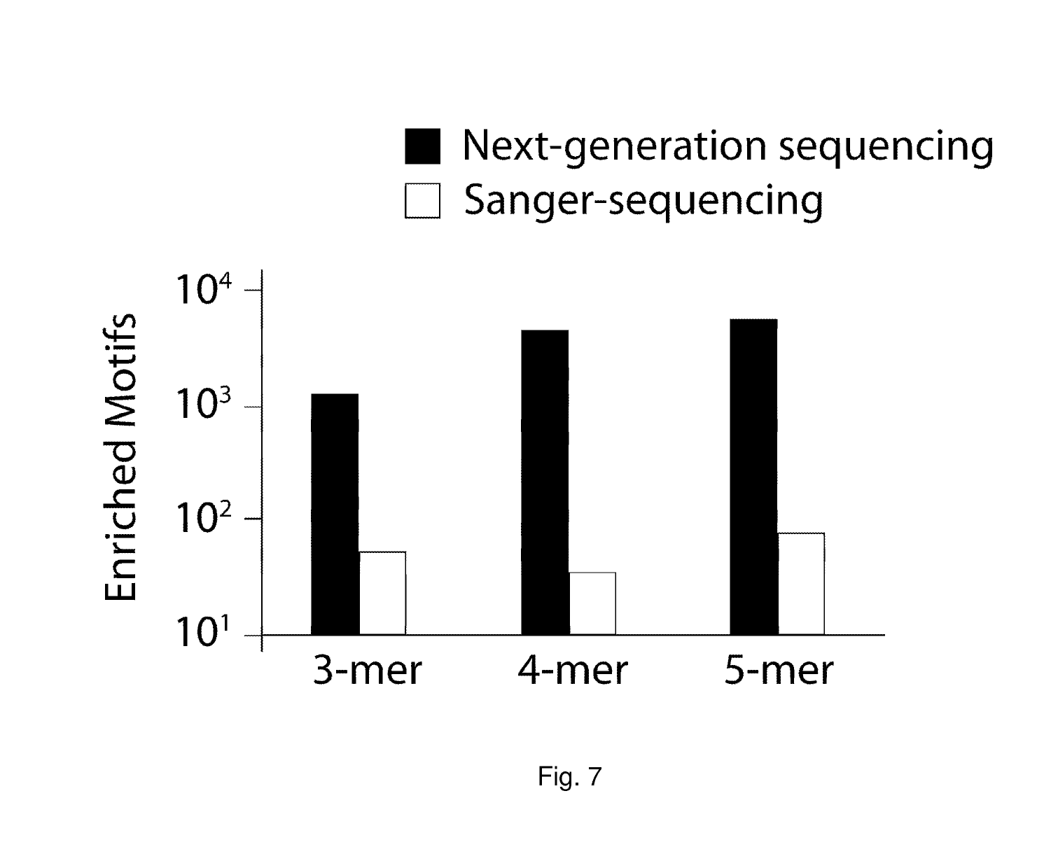

To determine phage diversity and candidate peptide motif enrichment, we calculated saturation curves from large-scale sequences produced from either each tissue and/or unselected library and showed that the coverage of phage particles present in these targeted tissues varied from 93.3 to 94.4% of the available predicted distinct total peptides in each tissue. The high coverage achieved for all tissues after the sequencing of 40,000 to 63,000 phage amplicons in this round of synchronous selection [29] strongly suggests that most of the diversity in these tissues has been covered (FIGS. 4A-D). In striking contrast to the targeted tissues, the sequencing of the non-selected library showed its predicted diversity, which was far from saturation post evaluation of 5×104 DNA sequences (FIG. 4E). Finally, we investigated whether this large dataset would allow the discrimination of more and/or longer motifs in the distinct tissues than previously reported [20]. Indeed, we observed that many more 3-mer, as well as 4-mer and 5-mer motifs, were revealed by next-generation sequencing (from 10- to 100-fold increment) in comparison to those observed in the more limited, Sanger-sequencing-derived dataset (FIG. 7). These results show that DNA deep-sequencing through next-generation approaches (i) is technically feasible and (ii) offers an unprecedented high-coverage of the displayed ligand repertoire. All phage sequences produced here are available online (Dias-Neto, et al., Next-Generation Phage Display: Integrating and Comparing Available Molecular Tools to Enable Cost-Effective High Throughput Analysis; PLoS ONE 4(12):8338, 2009).

### A Comparative Analysis of Time- and Cost-Effectiveness

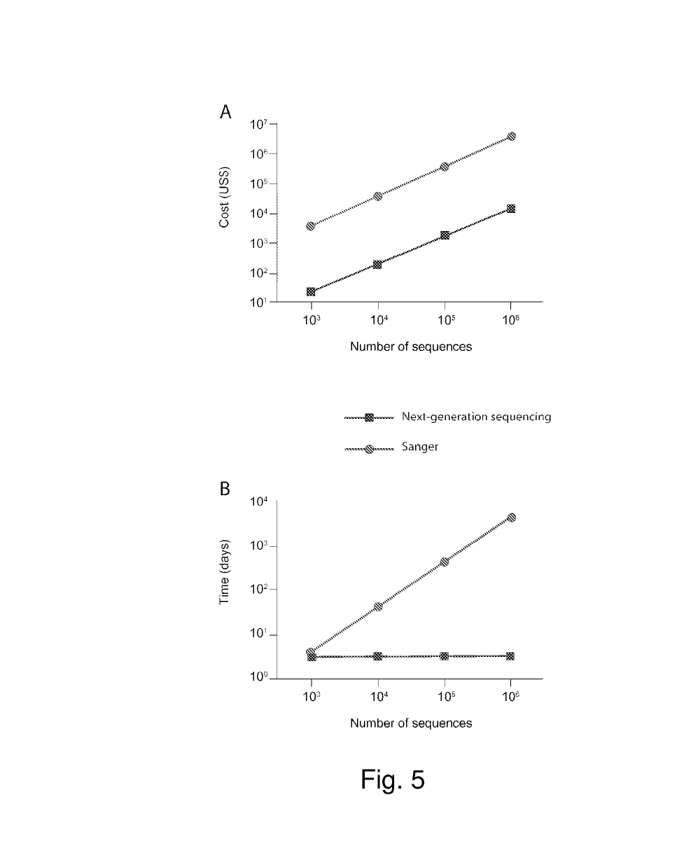

We performed a comparative analysis of time and cost required to reach from 103 to 106 sequences from a phage library by using both approaches (Table 4). Even with labor costs excluded from the calculation (i.e., only reagents and plastic-ware), there is a clear advantage of qPhage plus next-generation sequencing over TU-counting plus traditional Sanger-sequencing. Specifically, the generation of 103 to 106 sequences costs ˜250-fold less than the conventional methodology. This strong cost-effectiveness remains constant regardless of the increasing sequence number within this range (FIG. 5A). Moreover, whereas the generation of 103 sequences is only 0.25-fold faster, the generation of 104, 105, and 106 sequences will be 13-fold, 130-fold, and 1,300-fold faster, respectively. Therefore, in contrast to the high fixed-cost differential, the marked improvement in time-effectiveness becomes even more evident by the generation of larger number of sequences, which may actually be required to cover the full ligand diversity (FIG. 5B).

TABLE 4
Estimated time and costs required for generation of DNA sequences
with TU-counting versus pyrosequencing.

| TU-counts | | | 454-Pyrosequencing | | |
|---|---|---|---|---|---|
| | Time | Cost | | Time | Cost |
| Activity | required | (US$) | Activity | required | (US$) |

To reach 1,000 sequences

| | | | | | |
|---|---|---|---|---|---|
| K91 | 16 h | 1.84 | DNA | 1 h | 2.2 |
| infection, | | | extraction | | |
| plating and | | | PCR | 45 min | 2.3 |
| colony | | | Gel analysis | 45 min | 1.75 |
| growth* | | | and | | |
| Colony | 2.5 h | 0 | sample | | |
| picking | | | concentration | | |
| PCR | 24 h | 385.00 | DNA | 20 min | 1.00 |
| Gel | 3 h | 50 | quantification | | |
| electro- | | | Ligation of | 72 h | 13.00 |
| phoresis | | | adapters and | | |
| DNA | 72 h | 3,000 | DNA | | |
| se- | | | sequencing** | | |
| quencing** | | | TOTAL | 74.8 h | 20.25 |
| TOTAL | 93.5 h | 3,436.84 | | | |
| | (3.9 days) | | | | |

To reach 10,000 sequences

| | | | | | |
|---|---|---|---|---|---|
| K91 | 24 h | 18.4 | DNA | 1 h | 2.2 |
| infection, | | | extraction | | |
| plating and | | | PCR | 45 min | 2.3 |
| colony | | | Gel analysis | 45 min | 1.75 |
| growth* | | | and | | |
| Colony | 25 h | 0 | sample | | |
| picking | | | concentration | | |
| PCR | 240 h | 3850 | DNA | 20 min | 1.00 |
| Gel | 6 h | 100 | quantification | | |
| electro- | | | Ligation of | 72 h | 130.00 |
| phoresis | | | adapters and | | |
| DNA | 720 h | 30,000 | DNA | | |
| se- | | | sequencing** | | |
| quencing** | | | TOTAL | 74.8 h | 137.25 |
| TOTAL | 1,015 h | 33,968.4 | | | |
| | (42 days) | | | | |

To reach 100,000 sequences

| | | | | | |
|---|---|---|---|---|---|
| K91 | 36 h | 184 | DNA | 1 h | 2.2 |
| infection, | | | extraction | | |
| plating and | | | PCR | 45 min | 2.3 |
| colony | | | Gel analysis | 45 min | 1.75 |
| growth* | | | and | | |
| Colony | 250 h | 0 | sample | | |
| picking | | | concentration | | |
| PCR | 2400 h | 38,500 | DNA | 20 min | 1.00 |
| Gel | 12 h | 200 | quantification | | |
| electro- | | | Ligation of | 72 h | 1300 |
| phoresis | | | adapters and | | |
| DNA | 7200 h | 300,000 | DNA | | |
| se- | | | sequencing** | | |
| quencing** | | | TOTAL | 74.8 h | 1307.25 |
| TOTAL | 9,898 h | 338,884 | | | |
| | (412 | | | | |
| | days) | | | | |

To reach 1,000,000 sequences

| | | | | | |
|---|---|---|---|---|---|
| K91 | 54 h | 1,840 | DNA | 1 h | 2.2 |
| infection, | | | extraction | | |
| plating and | | | PCR | 45 min | 2.3 |
| colony | | | Gel analysis | 45 min | 1.75 |
| growth | | | and | | |
| Colony | 2500 h | 0 | sample | | |
| picking | | | concentration | | |
| PCR | 24000 h | 385,000 | DNA | 20 min | 1.00 |
| Gel | 24 h | 400 | quantification | | |
| electro- | | | Ligation of | 72 h | 1200.00 |
| phoresis | | | adapters and | | |
| DNA | 72,000 h | 3,000,000 | DNA | | |
| se- | | | se- | | |
| quencing** | | | quencing*** | | |
| TOTAL | 98,578 h | 3,387,240 | TOTAL | 74.8 h | 13007.25 |
| | (4,106 | | | | |
| | days) | | | | |

*Considering ideal bacterial densities in all plates, allowing the recovery of 250 colonies/plate and a cost of US$0.46/plate;
**Considering the availability of a dedicated DNA sequencer running 3.3 plates/day, at a cost of US$3.00/sample;
***The time required for this step is the same for the number of sequences given in this example or for a full 454 Titanium platform (1,00,000 reads). Cost of this step is proportional to the overall cost of US$13,000/1 million reads at the DNA sequencing Core facility at The University of Texas M. D. Anderson Cancer Center.

### Discussion

In this study, we address the two least efficient steps of phage display library selection: quantification and analysis of the displayed ligands. We introduce an integrated, robust, and readily available set of DNA-based molecular tools that will markedly improve combinatorial analysis in vitro and in vivo, and at an extremely low cost in labor and time.

Phage library quantification is currently scored through phage infection of host bacteria, serial dilution, and TU-counting (i.e., individual colony or plaque). In this process, phage recovery and library titer depend on a number of factors such as peptide-target affinity, bacterial toxicity of encoded peptides, viability of phage after panning and particle recovery, as well as inherent infection and replication properties of targeted phage clones. Indeed, certain phage encoded-peptide sequences may be non-accurately represented due to preferences in the bacteria codon-usage. Notably, molecular events of host binding, entry, and infection rates depend on the number of ligands displayed and on the nature of the hybrid fusion partner; these currently unquantifiable factors could affect infection and replication capabilities of particular phage clones and might influence the prospect of uncovering rare ligands and/or the true library size. Suggested methods, developed to overcome some of these technical challenges include bacterial infection-independent procedures, such as enzyme-linked immunosorbent assay (ELISA) with antibodies that bind specifically to the phage coat [24] and/or phage-DNA-based approaches such as quantitative-PCR [25]-[26]. However, biochemical approaches clearly still lack the required sensitivity when phage titers are low. Moreover, the concept of “high-throughput” pyrosequencing of phage display libraries has now evolved from a very complex protocol that yielded merely ˜102 amplicons [27]-[28] to a far easier technique that enables the sequencing of 106 amplicons as presented here. Finally, quantitative-PCR plus next-generation sequencing methodologies have not as yet been systematically compared to TU-counting plus Sanger sequencing in terms of speed, cost, and—most importantly—accuracy.

The next-generation phage display approach introduced here includes DNA-analysis of clones from the initial quantification steps to the final large-scale sequencing, which are relatively much faster and far less expensive. Replacement of bacterial overnight growth and TU-counting with DNA extraction and qPCR not only permits the quantification of non-infective/degraded phage, but it also yields phage homing results in only a few hours after tissue removal, simultaneously for dozens of samples in parallel. In real-time PCR phage quantification (qPhage), reproducible quantification was attained over a broad concentration range and was linear over at least eight orders of magnitude, far better than with the conventional approach and also much more sensitive than real-time PCR phage quantification reports [25]-[26].

Our qPhage strategy allows fast and precise validation of target tissue-specificity in the homing of selected phage or in large-scale evaluation of dozens of samples, which is particularly appealing for studies in vivo, including patients [29]-[30]. These goals are reachable with only small design modifications through simultaneous administration of multiple independent targeted phage particles in a single animal, followed by specific detection of each one with appropriate primers or probes in a multiplexed PCR. After homing validation, peptides shown after sequencing saturation of a number of vascular beds that appear to be specific to a particular target tissue, can be considered further as promising probes to be developed as agents for imaging and drug delivery in normal or tumor target sites.

Minor drawbacks remain. Host bacteria are still required for phage library generation and amplification between screening rounds; given the phage life cycle, this is unlikely to change. Another potential drawback is the need to re-clone the phage of interest (if desired), after its displayed peptide is determined. Nevertheless, deep sampling of the targeted peptide repertoire leads to a more reliable phage selection, and the regeneration of the selected particle(s) of interest can easily be accomplished with straightforward cloning protocols.

After titration with qPhage, the extracted DNA can be used directly for high-throughput determination of the displayed ligands (i.e., peptides or antibodies). In our tests, the generation of a large number of sequences allowed good coverage of the repertoire in all tissues studied, and included most of the sequences derived from conventional TU-counting. The availability of a larger nucleotide sequence dataset derived from high-throughput sequencing has also allowed the adoption of more stringent criteria to validate sequences. For example, we require that a peptide-encoding phage insert is accepted only if its sequence occurs at least twice, leading to the exclusion of singletons from the final dataset; such a “two-hit requirement” reduces sequencing errors in the final dataset of large-scale DNA sequencing-based approaches [31]-[32], and sharply increases our confidence in the displayed peptide list. This stringent criterion is not applicable to extremely diverse datasets in which repeats are not expected (i.e., first-round selection or library sequencing) or in reduced sequence datasets that are not large enough to cover the entire phage diversity; indeed, in such cases, the presence of sequencing errors or artifacts may be one of the factors potentially explaining why large-scale sequencing may not necessarily exhaust smaller sequence data sets.

As the new methodology proposed here is PCR-based (in contrast to a host bacteria-dependent approach), a number of potential advantages and disadvantages emerge. In general, a PCR-based approach is capable to reveal real binding peptides whose representation may be negatively impacted by the requirement of bacterial infection and multiplication. On the other hand, such PCR-based approach may acquire background noise due to errors in library construction or assembly of non-infective phage particles. Our analysis shows that most of the rejected sequences shown in Table 1 are derived from “empty” phage particles or amplification artifacts. However, the bioinformatic filters implemented here have allowed the prompt identification and discarding of these artifacts, revealing the relevant sequences in an unprecedented scale.

As the conventional phage display approach has long been validated, a central concern of this work was to evaluate whether any biases were introduced in the new steps that produced the amplicons to be sequenced. Our comparative analysis based on GC content and homopolymer frequency in the inserts, as well as codon usage, and residue or peptide frequencies and overlaps indicated that (i) there was no preferential amplification of certain inserts but (ii) both datasets share essentially the same sequence properties. As the sequencing of homopolymer-containing regions is a well-known limitation of the 454-Roche pyrosequencing platform used here, this issue was investigated in detail. As presented in the tables, the rejected 454 sequence-dataset contains more homopolymers than the accepted 454 sequence-set (chi-square test, P<0.001; Table 5) and the more abundant classes of homopolymer-containing sequences (frequency >3) appear to be somewhat under-estimated (Table 6). This suggests that insert sequences containing homopolymers >5 nt are under-estimated after 454-pyrosequencing. However, when all accepted sequences were evaluated (Table 3), we observed a non-statistically significant trend for a reduced frequency of homopolymers ≧5 in the 454-derived dataset when compared to the Sanger-derived sequence set (chi-square test, P=0.9955). The fact that both datasets are similar in terms of homopolymer-containing sequences is likely due to a simple fact. After PCR, each phage is amplified generating millions of copies of the original molecule. A certain percentage (˜15%) of the homopolymer-containing amplicons, will not be correctly sequenced by 454. However, due to the massive capability of this approach, enough molecules will still be correctly sequenced and represented in the final dataset. Thus both sequence sets (454-pyrosequencing and Sanger) will be similar when the distinct sets of homopolymer sizes are considered.

TABLE 5
Homopolymer-containing sequences in rejected- and accepted-
454-pyrosequencing datasets.*

| Homopolymer | 454-sequences | |
|---|---|---|
| size (nt) | Rejected set | Accepted set |
| 4 | 1128/3826 (29.5%) | 813/2847 (28.6%) |
| 5 | 821/3826 (21.5%) | 337/2847 (11.8%) |
| 6 | 459/3826 (12.0%) | 128/2847 (4.5%) |
| 7 | 184/3826 (4.8%) | 49/2847 (1.7%) |
| 8 | 94/3826 (2.5%) | 16/2847 (0.6%) |
| ≧4 | 2686/3826 (70.2%) | 1343/2847 (47.2%) |
| ≧5 | 1558/3826 (40.7%) | 530/2847 (18.6%) |

*The null-hypothesis that there is no significant difference between the rejected and accepted sets in terms of the homopolymer-containing sequences that they contain can be rejected based on a Chi-square test (P-value = 0.000001). However, one should also note that the fractions for 4-mers are very close, suggesting that this effect is noticeable for k-mers with k = 5 or greater.

TABLE 6
Homopolymers (≧5 nt) in rare, medium, or abundant frequency groups.

| | Accepted sequences | |
|---|---|---|
| Frequency groups | 454-pyrosequencing | Sanger-sequencing |
| Rare (1-2) | 582/3074 (18.9%) | 240/1296 (18.5%) |
| Medium (3-10) | 1527/8650 (17.6%) | 297/1070 (27.8%) |
| Abundant (>10) | 15024/175717 (8.55%) | 364/1188 (30.6%) |

To reinforce the similarity of both datasets, when sequences derived from both DNA sequencing methods (N=1645) or sequences exclusively found by 454-pyrosequencing (N=1202) were compared, we observe no significant differences in the frequency of homopolymers of all sizes (4 to 7 repeated bases). The comparison of homopolymer-containing inserts between these large groups and the group of sequences exclusively found by Sanger-sequencing is not informative, as it lacks precision due to its relatively small size (N=87, compared to >1000 for the other groups). However, it is interesting to note that the frequency of homopolymers in sequences found only by the Sanger method was higher for all classes of homopolymer sizes. This effect may be real, but is certainly small as we can see from the small size of this group (Table 7).

TABLE 7
Homopolymers in inserts found by each or both sequencing methods.

| Homopolymer | | 454-pyrosequencing | Sanger and 454- |
|---|---|---|---|
| size | Sanger only | only | pyrosequencing |
| (nt) | (N = 87) | (N = 1645) | (N = 1202) |
| 4 | 18/87 (20.7%) | 460/1645 (28.0%) | 353/1202 (29.4%) |
| 5 | 18/87 (20.7%) | 196/1645 (11.9%) | 141/1202 (11.7%) |
| 6 | 9/87 (10.3%) | 72/1645 (4.3%) | 56/1202 (4.6%) |
| 7 | 5/87 (5.7%) | 30/1645 (1.8%) | 19/1202 (1.6%) |
| 8 | 8/87 (0.9%) | 9/1645 (0.6%) | 7/1202 (0.6%) |
| ≧4 | 58/87 (66.7%) | 767/1645 (46.6%) | 576/1202 (47.9%) |
| ≧5 | 40/87 (46.0%) | 307/1645 (18.7%) | 223/1202 (18.6%) |

As noted above, the sequencing of homopolymers is an established technical issue for the pyrosequencing methodology. However, from the analysis presented here, we can conclude that this technical limitation has only had a very small effect on the universe of phage particles revealed by the large-scale approach. As the 454-method allowed a high coverage of the Sanger dataset for all tissues evaluated (ranging from 78.6 to 96.3%), and it also uncovered a significant fraction of peptides (25.3 to 97.7%) not revealed by the low-throughput Sanger-sequencing approach, we conclude that the benefits of this approach certainly compensate the known disadvantages and challenges of this particular sequencing platform. As demonstrated for other platforms (such as the SOLiD, Applied Biosystems), technical alternatives exist for the particular sequencing of homopolymeric-rich regions. In the future, the integrated approach presented here may eventually be chosen for use with alternative high-throughput sequencing approaches other than the one developed by 454-Roche.

The large sequence dataset presented here has covered over 90% of the phage diversity of all human tissues we investigated and has provided a high-confidence list of tissue-specific ligands. Sets of high-confidence tissue-specific peptides along with improved statistical analysis of longer motifs can be undertaken after the sequencing of phage DNA recovered from a large number of tissues, as well as from specific tissue samples recovered by micro-dissection from paraffin-embedded tissues. In fact, large-scale sequencing of naïve (unselected and unamplified) libraries may—for the first time—provide an accurate measurement of their size (i.e., number of unique sequences), a result allowing the empiric (rather than theoretical) demonstration of the true randomness of insert sequences. In this study, it should be noted that we used targeting peptides for validation, but there is no reason that antibodies would not be as effective. Indeed, one might speculate that the DNA-based approaches introduced in this study will eliminate the need for “helper” phage for phage antibody-display selection, and finally enable its in vivo application.

One technical aspect merits an additional brief commentary. Next-generation sequencing approaches are being improved constantly, and the newest chemistry platforms [such as SOLiD™ (Applied Biosystems) or Illumina Genome Analyzer (Illumina, Inc.)] may actually permit the generation of >100 million sequencing “reads” per run without the inherent challenge of homopolymer sequencing of this pyrosequencing platform. For both sequencing technologies, a major limitation is the short length of possible DNA analytes (<100 nucleotides), which may prove suitable for combinatorial phage libraries (displaying small peptides). Nevertheless, because the accuracy and cost-effectiveness of such methods has not been vetted, it remains to be determined whether other massive sequencing platforms may eventually replace the platform used here.

Technological advances have already brought about a new era for genomics, epigenomics, and transcriptome studies. We predict the same will happen for phage display analysis. We show that the integration of DNA-based quantification and large-scale sequencing methodology presented here produces unbiased data and allows the full determination of the whole pool of ligand sequences available after “n” rounds of selection. Our results show that in tandem qPhage quantification and next-generation DNA sequencing will set a new gold standard for phage display for accuracy, running time, diversity coverage, and cost-effectiveness. Overall, the enabling platform introduced and optimized in this work is superior to TU-counting plus Sanger sequencing. As such, it may become the method-of-choice for a broad range of phage-display applications in silico, in cells, and in vivo; this will be particularly the case if the extreme molecular diversity observed during large-scale screenings in patients is considered.

### REFERENCES

1. Barbas C F III, Burton D R, Scott J K, Silverman G J (2001) Phage Display: A Laboratory Manual. New York: Cold Spring Harbor Laboratory Press. 736 p.
2. O' Brien P M, Aitken R (2002) Antibody Phage Display: Methods and Protocols. Totowa: Humana Press. 576 p.
3. Clarkson T, Lowman H B (2004) Phage Display: A Practical Approach. Oxford: Oxford University Press. 360 p.
4. Sidhu S S (2005) Phage Display in Biotechnology and Drug Discovery. Boca Raton: CRC Press. 768 p.
5. Zacher A N 3rd, Stock C A, Golden J W 2nd, Smith G P (1980) A new filamentous phage cloning vector: fd-tet. Gene 9: 127-140.
6. Koivunen E, Wang B, Ruoslahti E (1995) Phage libraries displaying cyclic peptides with different ring sizes: ligand specificities of the RGD-directed integrins. Biotechnology (NY) 13: 265-270.
7. Pasqualini R, Koivunen E, Ruoslahti E (1997) Alpha v integrins as receptors for tumor targeting by circulating ligands. Nat Biotech 15: 542-546.
8. Arap W, Pasqualini R, Ruoslahti E (1998) Cancer treatment by targeted drug delivery to tumor vasculature in a mouse model. Science 279: 377-380.
9. Giordano R J, Cardó-Vila M, Landenranta J, Pasqualini R, Arap W (2001) Biopanning and rapid analysis of selective interactive ligands. Nat Med 7: 1249-1253.
10. Hajitou A, Trepel M, Lilley C E, Soghomonyan S, Alauddin M M, et al. (2006) A hybrid vector for ligand-directed tumor targeting and molecular imaging. Cell 125: 385-398.
11. Arap M A, Landenranta J, Mintz P J, Hajitou A, Sarkis A S, et al. (2004) Cell surface expression of the stress response chaperone GRP78 enables tumor targeting by circulating ligands. Cancer Cell 6: 275-284.
12. Kolonin M G, Saha P K, Chan L, Pasqualini R, Arap W (2004) Reversal of obesity by targeted ablation of adipose tissue. Nat Med 10: 625-632.
13. Marchiò S, Landenranta J, Schlingemann R O, Valdembri D, Wesseling P, et al. (2004) Aminopeptidase A is a functional target in angiogenic blood vessels. Cancer Cell 5: 151-162.
14. Zurita A J, Troncoso P, Cardó-Vila M, Logothetis C J, Pasqualini R, et al. (2004) Combinatorial screenings in patients: the interleukin-11 receptor alpha as a candidate target in the progression of human prostate cancer. Cancer Res 64: 435-439.
15. Nishimura S, Takahashi S, Kamikatahira H, Kuroki Y, Jaalouk D E, et al. (2008) Combinatorial targeting of the macropinocytotic pathway in leukemia and lymphoma cells. J Biol Chem 283: 11752-11762.
16. Staquicini F I, Dias-Neto E, Li J, Snyder E Y, Sidman R L, et al. (2009) Discovery of a functional protein complex of netrin-4, laminin gamma1 chain, and integrin alpha6beta1 in mouse neural stem cells. Proc Natl Acad Sci USA 106: 2903-2908.
17. Mintz P J, Cardo-Vila M, Ozawa M G, Hajitou A, Rangel R, et al. (2009) An unrecognized extracellular function for an intracellular adapter protein released from the cytoplasm into the tumor microenvironment. Proc Natl Acad Sci USA 106: 2182-2187.
18. Pentz R D, Cohen C B, Wicclair M, DeVita M A, Flamm A L (2005) Ethics guidelines for research with the recently dead. Nat Med 11: 1145-1149.
19. Pentz R D, Flamm A L, Pasqualini R, Logothetis C J, Arap W (2003) Revisiting ethical guidelines for research with terminal wean and brain-dead participants. Hastings Cent Rep 33: 20-26.
20. Arap W, Kolonin M G, Trepel M, Landenranta J, Cardo-Vila M, et al. (2002) Steps toward mapping the human vasculature by phage display. Nat Med 8: 121-127.
21. Margulies M, Egholm M, Altman W E, Attiya S, Bader J S, et al. (2005) Genome sequencing in microfabricated high-density picolitre reactors. Nature 437: 376-380.
22. Huse S M, Huber J A, Morrison H G, Sogin M L, Welch D M (2007) Accuracy and quality of massively parallel DNA pyrosequencing. Genome Biol 8: R143.
23. Moore M J, Dhingra A, Soltis P S, Shaw R, Farmerie W G, et al. (2006) Rapid and accurate pyrosequencing of angiosperm plastid genomes. BMC Plant Biol 6: 16.
24. Walter G, Konthur Z, Lehrach H (2001) High-throughput screening of surface displayed gene products. Comb Chem Highthr Scr 4: 193-205.
25. Ballard V L, Holm J M, Edelberg J M (2006) Quantitative PCR-based approach for rapid phage display analysis: a foundation for high throughput vascular proteomic profiling. Physiol Genomics 26: 202-208.
26. Jaye D L, Nolte F S, Mazzucchelli L, Geigerman C, Akyildiz A, et al. (2003) Use of real-time polymerase chain reaction to identify cell- and tissue-type-selective peptides by phage display. Am J Pathol 162: 1419-1429.
27. Rahim A, Coutelle C, Harbottle R (2003) High-throughput pyrosequencing of a phage display library for the identification of enriched target-specific peptides. Biotechniques 35: 317-324.
28. Rahim A A (2007) Pyrosequencing of phage display libraries for the identification of cell-specific targeting ligands. Meth Mol Biol 373: 135-146.
29. Kolonin M G, Sun J, Do K A, Vidal C I, Ji Y, et al. (2006) Synchronous selection of homing peptides for multiple tissues by in vivo phage display. FASEB J 20: 979-981.
30. Krag D N, Shukla G S, Shen G P, Pero S, Ashikaga T, et al. (2006) Selection of tumor-binding ligands in cancer patients with phage display libraries. Cancer Res 66: 7724-7733.
31. Ojopi E P, Oliveira P S, Nunes D N, Paquola A, DeMarco R, et al. (2007) A quantitative view of the transcriptome of Schistosoma mansoni adult-worms using SAGE. BMC Genomics 8: 186.
32. Guerfali F Z, Laouini D, Guizani-Tabbane L, Ottones F, Ben-Aissa K, et al. (2008) Simultaneous gene expression profiling in human macrophages infected with Leishmania major parasites using SAGE. BMC Genomics 9: 238.
33. Langley R R, Ramirez K M, Tsan R Z, Van Arsdall M, Nilsson M B (2003) Tissue-specific microvascular endothelial cell lines from H-2K(b).-tsA58 mice for studies of angiogenesis and metastasis. Cancer Res 63: 2971-2976.
34. Colwell R K (2006) EstimateS: Statistical estimation of species richness and shared species from samples (Version 8.0).
35. Chao A (1987) Estimating the population size for capture-recapture data with unequal catchability. Biometrics 43: 783-791.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims. The entire contents of any reference that is referred to herein are hereby incorporated by reference.

1. A method comprising
obtaining a sample of a primate target organ or tissue of interest, wherein the sample is from a primate subject to whom a library of ligand-encoding phage has been administered; and
quantifying by quantitative real time PCR phage content from the sample.

2. The method of claim 1, wherein the obtaining and quantifying does not require bacteria.

3. The method of claim 1, further comprising determining nucleotide sequence information for at least one ligand encoded by phage in the sample.

4. The method of claim 3, wherein the determining comprises determining through use of a fluorescent nucleotide sequencing platform.

5. The method of claim 3, wherein the determining comprises determining through use of high throughput sequencing.

6. The method of claim 5, wherein the high throughput sequencing is next generation sequencing.

7. The method of claim 5, wherein the high throughput sequencing is next generation pyrosequencing.

8. The method of claim 3, wherein at least 10³ sequences are determined in a single run of sequencing.

9. The method of claim 3, wherein the determining comprises determining through use of a non-fluorescent nucleotide sequencing platform.

10. The method of claim 1, wherein encoded ligands in the library are peptide ligands.

11. The method of claim 10, wherein encoded ligands in the library are peptides 3-100, 5-20, 5-15, 6-10 or 7-9 amino acids in size.

12.-15. (canceled)

16. The method of claim 10, wherein encoded ligands in the library are cyclic peptides.

17. The method of claim 1, wherein encoded ligands are antibodies or fragments thereof.

18. The method of claim 17 wherein the antibodies or fragments thereof are selected from the group consisting of antibody-like molecules, Fc portions, Fab's, ScFv's, single domain antibodies, and combinations thereof.

19. The method of claim 17, wherein the antibodies are monoclonal.

20. The method of claim 1, wherein ligand-encoding sequences in two or more phage of the library are flanked by amplifiable primer sequences such that a single set of primers used in the quantitative real time PCR will amplify ligand-encoding sequences from two or more phage within the library.

21. The method of claim 1, wherein the target organ or tissue of interest is or comprises bone marrow, breast, ovary, coronary artery, fat, muscle, skin, lymph node, heart, spleen, lung, kidney, dura mater, adrenal gland, testis, prostate, bladder, brain, thyroid, aorta, esophagus, stomach, duodenum, pancreas, gall bladder, liver, large bowel, small bowel, stem cells, stromal cells, or endothelial cells.

22. The method of claim 21, wherein the target organ or tissue of interest is or comprises at least two of bone marrow, breast, ovary, coronary artery, fat, muscle, skin, lymph node, heart, spleen, lung, kidney, dura mater, adrenal gland, testis, prostate, bladder, brain, thyroid, aorta, esophagus, stomach, duodenum, pancreas, gall bladder, liver, large bowel, small bowel, stem cells, stromal cells, or endothelial cells.

23. The method of claim 1, wherein the target organ or tissue of interest is selected from the group consisting of bone marrow, breast, ovary, coronary artery, fat, muscle, skin, epidermis, dermis, subcutis, lymph node, heart, spleen, lung, kidney, renal glomeruli, dura mater, adrenal gland, testis, prostate, bladder, brain, cerebellum, cerebrum, thyroid, aorta, esophagus, stomach, duodenum, pancreas, pancreatic islet, gall bladder, liver, large bowel, small bowel, stem cells, stromal cells, endothelial cells, and combinations thereof.

24. The method of claim 23, wherein the target organ or tissue of interest is selected from the group consisting of epidermis, dermis, subcutis, and combinations thereof.

25. The method of claim 23, wherein the target organ or tissue of interest is selected from the group consisting of cerebellum, cerebrum, dura mater, and combinations thereof.

26. The method of claim 1, wherein the sample of a target organ or tissue of interest is a tumor sample.

27. The method of claim 26, wherein the tumor sample is or comprises a primary tumor sample.

28. The method of claim 26, wherein the tumor sample is or comprises a metastatic tumor sample.

29. The method of claim 1, wherein the primate subject is a non-human primate subject.

30. The method of claim 1, wherein the primate subject is a human subject.

31. The method of claim 30, wherein the human subject is an end-of-life patient, a trauma patient, a brain-dead patient or a patient having a surgical tumor resection.

32.-34. (canceled)

35. The method of claim 1, wherein the obtaining comprises taking or receiving the sample from a surgical biopsy sample.

36. The method of claim 1, wherein the isolating comprises obtaining the sample from an autopsy.

37. The method of claim 36, wherein the sample comprises intact organ or tissue.

38. The method of claim 1, wherein the step of obtaining is performed at least 24 h after administration of the library to the human subject.

39. The method of claim 1, wherein the quantitative real time PCR is compared to a reference sample.

40. The method of claim 1, wherein the quantifying utilizes methodology that quantifies non-infective phage particles present in the sample.

41. The method of claim 1, wherein the steps of obtaining and quantifying are completed within a time period not longer than 1 hours.

42. The method of claim 3, further comprising determining that identical ligand sequence information is obtained from at least two phage in the sample.

43. In a method of identifying ligands that target one or more organs or tissues by in vivo phage display, the improvement that comprises one or more of:
a) obtaining an organ or tissue sample from an autopsy;
b) quantifying ligands encoded by phage in the absence of bacteria;
c) quantifying ligands encoded by phage by quantitative real time PCR;
d) identifying ligands present at low abundance in the one or more target organs or tissues; and
e) obtaining sequence information by next generation pyrosequencing.
